(19) United States
(12) Patent Application Publication
(10) Pub. No.: US 2006/0014752 A1
(43) Pub. Date: Jan. 19, 2006

(54) Heterocyclic anti-epileptogenic agents and methods of use thereof

(75) Inventors: Donald F. Weaver, Halifax (CA); Allyson J. Campbell, Tonbridge (GB)

Correspondence Address: LAHIVE & COCKFIELD, LLP., 28 STATE STREET, BOSTON, MA, 02109, US

(73) Assignee: Queen's University at Kingston, Kingston (CA)

(21) Appl. No.: 11/099,232

(22) Filed: Apr. 4, 2005

Related U.S. Application Data
(63) Continuation of application No. 10/222,141, filed on Aug. 16, 2002, now abandoned.
Application No. 10/222,141 is a continuation-in-part of application No. PCT/CA02/00773, filed on May 27, 2002.
(60) Provisional application No. 60/293,495, filed on May 25, 2001.

(51) Int. Cl. A61K 31/497

(52) U.S. Cl. 514/252.1

(57) ABSTRACT

Methods and compounds, such as β-heterocyclic-β-amino acids, useful for the inhibition of epileptogenesis are disclosed. Methods for preparing and using the β-heterocyclic-β-amino acids of the invention are also described.

### RELATED APPLICATIONS

This application is a Continuation of application Ser. No. 10/222,141, filed on Aug. 16, 2002, which is a Continuation-in-part of application PCT/CA02/00773, filed on May 27, 2002, which claims the benefit of Provisional application 60/293,495 under 35 USC 119(e), filed on May 25, 2001. The entire contents of each of the foregoing patent applications are expressly incorporated herein by reference in their entirety.

### BACKGROUND OF THE INVENTION

Epilepsy is a serious neurological condition, associated with seizures, that affects hundreds of thousands of people worldwide. Clinically, a seizure results from a sudden electrical discharge from a collection of neurons in the brain. The resulting nerve cell activity is manifested by symptoms such as uncontrollable movements.

A seizure is a single discrete clinical event caused by an excessive electrical discharge from a collection of neurons through a process termed “ictogenesis.” As such, a seizure is merely the symptom of epilepsy. Epilepsy is a dynamic and often progressive process characterized by an underlying sequence of pathological transformations whereby normal brain is altered, becoming susceptible to recurrent seizures through a process termed “epileptogenesis.” While it is believed that ictogenesis and epileptogenesis have certain biochemical pathways in common, the two processes are not identical. Ictogenesis (the initiation and propagation of a seizure in time and space) is a rapid and definitive electrical/chemical event occurring over seconds or minutes. Epileptogenesis (the gradual process whereby normal brain is transformed into a state susceptible to spontaneous, episodic, time-limited, recurrent seizures, through the initiation and maturation of an “epileptogenic focus”) is a slow biochemical or histological process which generally occurs over months to years. Epileptogenesis is a two phase process. Phase 1 epileptogenesis is the initiation of the epileptogenic process prior to the first seizure, and is often the result of stroke, disease (e.g., meningitis), or trauma, such as an accidental blow to the head or a surgical procedure performed on the brain. Phase 2 epileptogenesis refers to the process during which a brain that is already susceptible to seizures, becomes still more susceptible to seizures of increasing frequency or severity. While the processes involved in epileptogenesis have not been definitively identified, some researchers believe that upregulation of excitatory coupling between neurons, mediated by N-methyl-D-aspartate (NMDA) receptors, is involved. Other researchers implicate downregulation of inhibitory coupling between neurons, mediated by gamma-amino-butyric acid (GABA) receptors, pre- or post-synaptically.

Although epileptic seizures are rarely fatal, large numbers of patients require medication to avoid the disruptive, and potentially dangerous, consequences of seizures. In many cases, medication is required for extended periods of time, and in some cases, a patient must continue to take prescription drugs for life. Furthermore, drugs used for the management of epilepsy have side effects associated with prolonged usage, and the cost of the drugs can be considerable.

A variety of drugs are available for the management of epileptic seizures, including older anticonvulsant agents such as phenytoin, valproate and carbamazepine (ion channel blockers), as well as newer agents such as felbamate, gabapentin, and tiagabine. β-Alanine has been reported to have anticonvulsant activity, as well as NMDA inhibitory activity and GABAergic stimulatory activity, but has not been employed clinically. Currently available accepted drugs for epilepsy are anticonvulsant agents, where the term “anticonvulsant” is synonymous with “anti-seizure” or “anti-ictogenic;” these drugs can suppress seizures by blocking ictogenesis, but it is believed that they do not influence epilepsy because they do not block epileptogenesis. Thus, despite the numerous drugs available for the treatment of epilepsy (i.e., through suppression of the convulsions associated with epileptic seizures), there are no generally accepted drugs for the treatment of the pathological changes which characterize epileptogenesis. There is no generally accepted method of inhibiting the epileptogenic process and there are no generally accepted drugs recognized as anti-epileptogenic.

### SUMMARY OF THE INVENTION

This invention relates to methods and compounds useful for the treatment of epileptogenesis-associated conditions such as, for example, epilepsy.

In one embodiment, the invention pertains to a method for inhibiting epileptogenesis in a subject. The method includes administering to the subject an effective amount of an anti-epileptogenic agent, such as, for example, β-heterocyclic-β-amino acid, or a compound of Formula I: 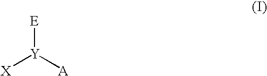
wherein X is a heterocyclic moiety, E is a hydrogen bond donor, Y is a connecting moiety, and A is an hydrogen bond acceptor, or a pharmaceutically acceptable salt, ester, N-substituted analog, C-substituted analog, bioisostere, or prodrug thereof.

In another embodiment, the invention pertains to a method for treating a subject suffering from an epileptogenesis-associated condition. The method includes administering to the subject an effective amount of an anti-epileptogenic agent, such as, for example, a β-heterocyclic-β-amino acid or a compound of Formula I.

The invention also pertains to a method for treating convulsions in a subject comprising administering to said subject an effective amount of an anti-epileptogenic agent (e.g., a β-heterocyclic-β-amino acid or a compound of Formula I).

In yet another embodiment, the invention pertains, at least in part, to pharmaceutical compositions, comprising a therapeutically effective amount of an anti-epileptogenic agent and a pharmaceutical acceptable carrier, wherein said anti-epileptogenic agent is of the Formula (II): 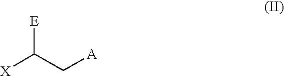
wherein X is a heterocyclic moiety, E is a hydrogen bond donor, and A is an hydrogen bond acceptor, or a pharmaceutically acceptable salt, ester, N-substituted analog, C-substituted analog, bioisostere, or prodrug thereof.

In a further embodiment, the invention pertains, at least in part, to a method of diagnosing an epileptogenesis-associated condition in a subject. The method includes administering an anti-epileptogenic agent (e.g., a compound of Formula I), labeled with a detectable marker to the subject; and measuring increased binding of the compound to the NMDA receptors of the neurons of the subject's brain.

In yet another embodiment, the invention pertains, at least in part, to a method of diagnosing an epileptogenesis-associated state. The method includes administering an anti-epileptogenic agent (e.g., a compound of Formula 1) labeled with a detectable marker to a subject; and measuring decreased binding of the compound to the GABA receptors of the neurons of the subject's brain.

These and other objects, features, and advantages of the invention will be apparent from the following description and claims.

### DETAILED DESCRIPTION OF THE INVENTION

The present invention pertains to methods and agents useful for the treatment of epilepsy and convulsive disorders, for inhibition of epileptogenesis, and for inhibition of ictogenesis; and to methods for preparing the anti-epileptogenic agents of the invention. The invention further pertains to pharmaceutical compositions for treatment of epileptogenic conditions, and to kits including the anti-epileptogenic agents of the invention.

In one embodiment, the invention pertains to a method for inhibiting epileptogenesis in a subject. The method includes administering to the subject an effective amount of an anti-epileptogenic agent, such as, for example a β-heterocyclic-β-amino acid, e.g., a β-heteroaromatic-β-amino acid.

The invention also pertains to methods for treating a subject suffering from an epileptogenesis-associated condition. The method includes administering to the subject an effective amount of an anti-epileptogenic agent (e.g., a β-heterocyclic-β-amino acid, e.g., a β-heteroaromatic-β-amino acid).

In another embodiment, the invention also includes a method for treating convulsions (e.g., seizures) in a subject. The method includes administering to a subject an effective amount of an anti-epileptogenic agent (e.g., a β-heterocyclic-β-amino acid, e.g., a β-heteroaromatic-β-amino acid).

The term “inhibiting epileptogenesis” includes both partial and complete reversal of epileptogenesis. It also includes prevention of epileptogenesis or a decrease or slowing in the rate of epileptogenesis (e.g., a partial or complete stop in the rate of epileptogenic transformation of the brain or central nervous system tissue). It also includes any inhibition or slowing of the rate of the biochemical processes or events which take place during Phase 1 or Phase 2 epileptogenesis and leads to epileptogenic changes in tissue, i.e., in tissues of the central nervous system (CNS), e.g., the brain. Examples of processes in pathways associated with epileptogenesis, which may be inhibited by the compounds of the invention, are discussed in more detail, infra. It also includes the prevention, slowing, halting, or reversing the process of epileptogenesis, i.e., the changes in brain tissue which result in epileptic seizures.

The term “convulsive disorder” or “convulsive condition” according to the invention includes conditions wherein a subject suffers from convulsions. Convulsive disorders include, but are not limited to, epilepsy, ictogenesis, epileptogenesis, and non-epileptic convulsions, and convulsions due to administration of a convulsive agent or trauma to the subject. The term “epileptogenesis-associated disorders” includes disorders of the central and peripheral nervous system which may advantageously be treated by the compounds of the invention. In an advantageous embodiment, the nervous system disorders are disorders associated or related to the process or the results of epileptogenic transformation of the brain or other nervous tissue. Examples of epileptogenesis-associated disorders include, but are not limited to, epilepsy, head trauma, stroke, schizophrenia, multiple sclerosis, amyotrophic lateral sclerosis, psychoses, cerebral ischemia, Huntington's chorea, motor neuron disease, Alzheimer's disease, dementia and other disorders (in humans or animals) in which excessive activity of NMDA receptors is a cause, at least in part, of the disorder (see, e.g., Schoepp et al., Eur. J. Pharmacol. 203:237-243 (1991); Leeson et al., J. Med. Chem. 34:1243-1252 (1991); Kulagowski et al., J. Med. Chem. 37:1402-1405 (1994); Mallarno et al., J. Med. Chem. 37:4438-4448 (1994); and references cited therein). Pain and anxiety are also known to be associated with excessive activity of NMDA receptors.

The terms “treatment,” “treating,” or “treat,” include the administration of an agent (e.g., an anti-epileptogenic agent) to a subject, who has a disease or disorder, a symptom of a disease or disorder, or is at risk of suffering from the disease or disorder in the future, such that the disease or disorder (or at least one symptom of the disease or disorder) is cured, healed, prevented, alleviated, relieved, altered, remedied, ameliorated, improved or otherwise affected, preferably in an advantageous manner. Agents include, but are not limited to, anti-epileptogenic agents (e.g., β-heterocyclic-β-amino acids).

The term “subject” includes animals susceptible to epileptogenesis or capable of suffering from epileptogenesis-associated states, such as warm-blooded animals, more preferably a mammal, including, e.g., non-human animals such as rats, mice, cats, dogs, sheep, horses, cattle, in addition to humans. In a preferred embodiment, the subject is a human.

The language “effective amount” of the compound is that amount necessary or sufficient to treat or prevent an epileptogenesis-associated state, e.g., to prevent the various symptoms of an epileptogenesis-associated state. The effective amount can vary depending on such factors as the size and weight of the subject, the type of illness, or the particular anti-epileptogenic agent. For example, the choice of the anti-epileptogenic agent can affect what constitutes an “effective amount.” One of ordinary skill in the art would be able to study the aforementioned factors and make the determination regarding the effective amount of the anti-epileptogenic agent without undue experimentation.

The term “anti-epileptogenic agent” includes agents which are capable of inhibiting epileptogenesis, e.g., suppressing the uptake of synaptic GABA (e.g., blocking GABA transporters, e.g. GAT-1, GAT-2 or GAT-3), depressing glutamatergic excitation (e.g., interacting with an NMDA receptor, e.g. at the strychnine-insensitive glycine co-agonist site), binding to a GABA receptor (e.g. GABA_{A}), altering (e.g., increasing or suppressing) the metabolism of GABA (e.g., via inhibition of GABA transaminase). Examples of anti-epileptogenic agents include β-heterocyclic-β-amino acids, e.g., β-heteroaromatic-β-amino acids, and pharmaceutically acceptable salts or esters, N-substituted analogs, C-substituted analogs, bioisosteres, and prodrugs thereof.

Other anti-epileptogenic agents of the invention include compounds of the Formula: 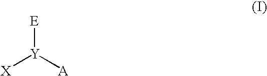
wherein:
X is a heterocyclic moiety;
Y is a connecting moiety;
E is a hydrogen bond donor; and
A is an hydrogen bond acceptor,
and pharmaceutically acceptable salts or esters, N-substituted analogs, C-substituted analogs, bioisosteres, and prodrugs thereof.

The term “heterocyclic moiety” (“X”) includes both saturated and unsaturated heterocyclic rings. The heterocyclic moiety may be lipophilic and may be substituted with any substituent that allows the anti-epileptogenic agent to perform its intended function. Furthermore, the heterocyclic moiety may be stereochemically rigid and may contain, for example, one or more aromatic rings. The heterocyclic moiety also may comprise carbocyclic rings either bridged or fused to a heteroaromatic ring. In an embodiment, the heterocyclic moiety includes rings such as, for example, pyrrolidine, oxolane, thiolane, piperidine, piperazine, morpholine, lactones, lactams, azetidinones, pyrrolidinones, sultams, sultones, and the like.

Other examples of heterocyclic moieties include monocyclic heteroaryls such as, for example, thienyl, pyrrolyl, pyrimidyl, pyrazinyl, pyrazolyl, oxazolyl, isooxazolyl, thiazolyl, isothiazolyl, imidazolyl, and furanyl.

In another embodiment, the heterocyclic moiety is multicyclic or polycyclic. The rings of the multicyclic or polycyclic heterocyclic moiety may be fused or bridged. In an embodiment, one of the bridged rings of the multicyclic heterocyclic moiety is phenyl (e.g., when at least one other ring of the polycyclic heterocyclic moiety is heterocyclic (e.g., thienyl, pyrrolyl, pyrimidyl, pyrazinyl, pyrazolyl, oxazolyl, isooxazolyl, thiazolyl, isothiazolyl, imidazolyl, or furanyl). In an embodiment, the bridged heterocyclic moiety is isooxazolylphenyl (e.g., an isooxazolyl ring bound to a phenyl ring).

In another further embodiment, the multicyclic (e.g., bicyclic, tricyclic, etc.) heterocyclic moiety comprises one or more fused rings. In an embodiment, at least one of the fused rings is aromatic. In another, two or more of the rings in the fused ring system are aromatic. Examples of multicyclic fused ring heterocyclic moieties include, but are not limited to, benzothiazolonyl, indolonyl, benzooxoazolinyl, benzothiophenyl, benzofuranyl, quinolinyl, isoquinolinyl, benzodioxazolyl, benzoxazolyl, benzothiazolyl, benzoimidazolyl, methylenedioxyphenyl, ethylenedioxyphenyl, indolyl, purinyl, and deazapurinyl.

Furthermore, each of the heterocyclic moieties described above, may be substituted with any substituent that allows the anti-epileptogenic agent to perform its intended function. Examples of substituents include, but are not limited to, alkyl (e.g., methyl, ethyl, propyl, butyl, etc.), alkenyl, alkynyl, halogen (e.g., fluorine, chlorine, bromine, iodine, etc.), hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, azido, heterocyclyl, or an aromatic or heteroaromatic moiety.

According to the invention, the term “hydrogen bond donor” (“E”) includes any moiety which is capable of being a hydrogen bond donor, such that the anti-epileptogenic agent is capable of performing its intended function. It also includes prodrugs of agents which are capable of being converted to the active form in vivo. Examples of hydrogen bond donors include, for example, NR²R³, OH, and SH, wherein R² and R³ are each independently hydrogen, alkyl, alkenyl, alkynyl, cycloalkyl, aryl, alkylaryl (e.g., benzyl and 1- or 2-phenethyl, i.e., α-methylbenzyl), alkylcarbonyl, arylcarbonyl (e.g., benzoyl), alkoxycarbonyl, or aryloxycarbonyl (provided that at least one of R² and R³ is hydrogen). In one embodiment, the hydrogen bond donor is NH₂, OH, or SH. In an advantageous embodiment, the hydrogen bond donor is NH₂.

According to the invention, the term “hydrogen bond acceptor” (“A”) includes any moiety which is capable of forming an electrostatic bond with a hydrogen atom of a hydrogen bond donor, such that the anti-epileptogenic agent is capable of performing its intended function. It also includes prodrugs of agents which are capable of being converted to the active form in vivo. In a preferred embodiment, hydrogen bond acceptors include anionic moieties, including moieties having a free electron pair, such as CO₂H (and esters thereof), sulfates, sulfonates, phosphonates, ketones, OH, and SH. A preferred hydrogen bond acceptor is CO₂H.

The term “anionic moiety” includes moieties which are either anionic under physiological conditions, polar, or chosen such that they allow the anti-epileptogenic agent to perform its intended function. Pharmaceutically acceptable salts of anionic moieties as well as their protonated forms are also included. Furthermore, prodrugs are also included, wherein a moiety may be converted to its active, or more active form once administered to a subject. Examples of prodrugs include esters which can be converted to carboxylate groups in vivo. Examples of anionic moieties include, but are not limited to, carboxylate (e.g., carboxylic acids), sulfate, sulfonate, sulfinate, nitrates, nitrites, sulfamate, phosphate, phosphonate, tetrazolyl, phosphinate, phosphorothioate, or functional equivalents thereof. Advantageous anionic moieties include carboxylate, carboxylic acids, and prodrugs thereof. “Functional equivalents” of anionic groups are intended to include bioisosteres, e.g., bioisosteres of a carboxylate group. Bioisosteres encompass both classical bioisosteric equivalents and non-classical bioisosteric equivalents. Classical and non-classical bioisosteres are known in the art (see, e.g., Silverman, R. B. The Organic Chemistry of Drug Design and Drug Action, Academic Press, Inc.: San Diego, Calif., 1992, pp. 19-23).

The term “connecting moiety” (“Y”) includes moieties which connect (e.g., through covalent bonds) each of the hydrogen bond acceptor, the hydrogen bond donor, and the heterocyclic moieties. In an embodiment, the connecting moiety comprises 1 to 20 atoms; and in a preferred embodiment, the connecting moiety comprises or consists of 1 to 6 carbon atoms (with the appropriate number of hydrogens). In another embodiment, the connecting moiety is selected such that the anti-epileptogenic agent of the invention is capable of performing its intended function, e.g., inhibiting epileptogenesis, treating nervous system disorders, antagonizing the NMDA receptor, suppressing uptake of synaptic GABA, etc. In another embodiment, the connecting moiety is selected such that the anti-epileptogenic compound of the invention is capable of being transported through the blood brain barrier. In one embodiment, the connecting moiety is comprised of from one to five carbon atoms, optionally substituted with hydrogen or another substituent which allows the agent to perform its intended function. In a further embodiment, the connecting moiety is alkyl, e.g., selected such that the resulting anti-epileptogenic agent is a β-amino acid.

In one embodiment, the anti-epileptogenic agent of the invention is of the Formula (II):

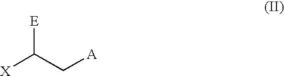

In a further preferred embodiment, the anti-epileptogenic agent of the invention is a β-amino-β-heterocyclic-1-propionic acid of the Formula (IIa): 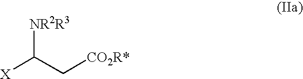
wherein:
R² and R³ are each independently hydrogen, alkyl, alkenyl, alkynyl, cycloalkyl, aryl, alkylaryl (e.g., benzyl and 1- or 2-phenethyl, i.e., α-methylbenzyl), alkylcarbonyl, arylcarbonyl (e.g., benzoyl), alkoxycarbonyl, or aryloxycarbonyl;
X is a heterocyclic moiety; and
R* is a substituted or unsubstituted alkyl moiety, a substituted or unsubstituted aryl moiety, a hydrogen, or a physiologically acceptable cation.

Examples of prodrugs include moieties which can be converted in vivo to the agents of the invention (see, e.g., R. B. Silverman, 1992, cited above, Chp. 8). Such prodrugs can be used to alter the biodistribution (e.g., to allow compounds that would not typically cross the blood-brain barrier to cross the blood-brain barrier) or the pharmacokinetics of the therapeutic compound. For example, anionic moieties (e.g., a carboxylate, sulfonate, etc.) can be esterified (e.g., with a methyl group or a phenyl group) to yield a carboxylate or sulfonate ester. When the carboxylate or sulfonate ester is administered to a subject, the ester is cleaved, enzymatically or non-enzymatically, to yield the anionic moiety. Such an ester can be cyclic (e.g., a lactone or sultone) or two or more anionic moieties may be esterified through a linking group. An anionic moiety can be esterified with groups (e.g., acyloxymethyl esters) which are cleaved to reveal an intermediate compound which subsequently decomposes to yield the active compound. Alternatively, an anionic moiety can be esterified to a group that is actively transported in vivo, or that is selectively taken up by target organs. The ester can be selected to allow specific targeting of the therapeutic moieties to particular organs. In another embodiment, the prodrug is a reduced form of an anionic group, e.g., a carboxylate or sulfonate, e.g., an alcohol or thiol, which is oxidized in vivo to the therapeutic compound.

Examples of anti-epileptogenic agents of Formula II, include but are not limited to, 3-(benzo[d]thiophen-3-yl)-3-aminopropionic acid, 3-(benzo[d]furan-2-yl)-3-aminopropionic acid, 3-(benzo[d]dioxolan-5-yl)-3-aminopropionic acid, 3-(quinolin-2-yl)-3-aminopropionic acid, 3-(2-chloroquinolin-3-yl)-3-aminopropionic acid, 3-(benzo[e]dioxan-6-yl)-3-aminopropionic acid, 3-(indol-5-yl)-3-aminopropionic acid, 3-(2-methylindol-5-yl)-aminopropionic acid, 3-(isoquinolin-4-yl)-3-aminopropionic acid, 3-(quinolin-3-yl)-3-aminopropionic acid, 3-(benzo[d]thiazolinon-5-yl)-3-aminopropionic acid, 3-(4-hydroxy-3-isoxazol-5-ylphenyl)-3-aminopropionic acid, and pharmaceutically acceptable salts or esters, N-substituted analogs, C-substituted analogs, bioisosteres, and prodrugs thereof.

Examples of anti-epileptogenic agents also include those compounds illustrated in the accompanying Examples as well as the following Table.

TABLE 1
Exemplary Compounds of the Invention

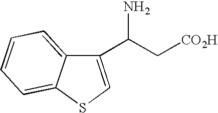

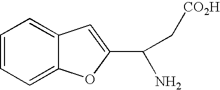

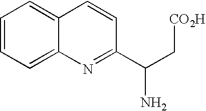

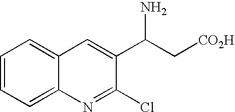

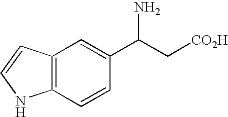

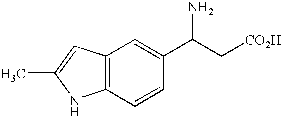

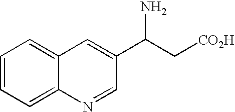

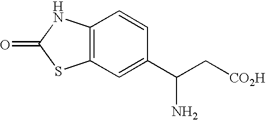

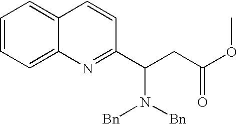

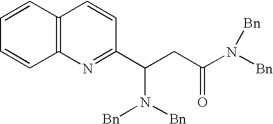

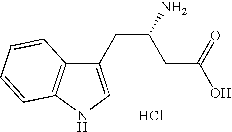

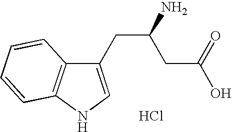

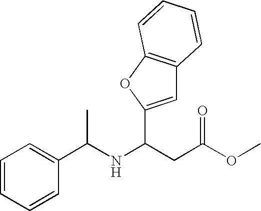

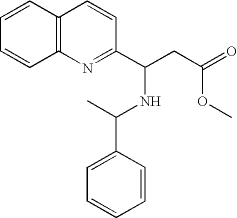

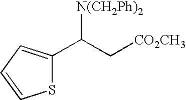

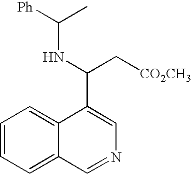

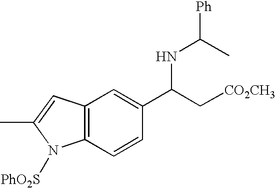

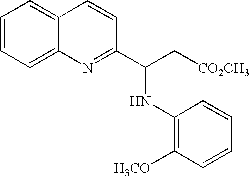

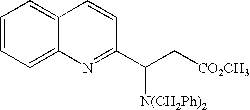

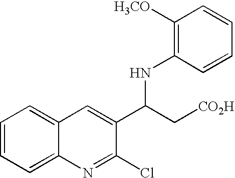

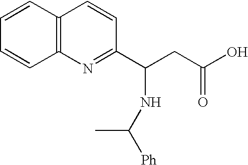

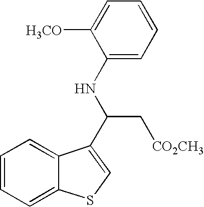

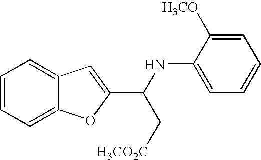

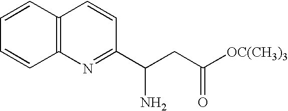

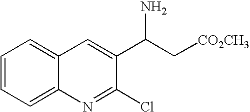

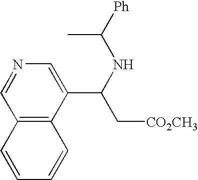

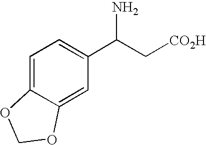

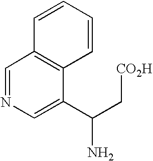

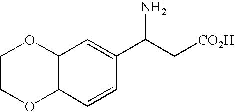

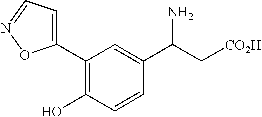

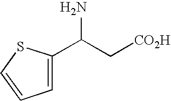

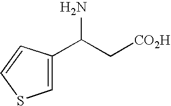

The heteroaryl groups represented in the example compounds above are therefore within the invention, i.e., those heteroaryl groups may be “X” in any Formula herein. Also within the scope of the present invention are prodrugs and pharmaceutically acceptable salts of the compounds in Table 1.

The β-heteroaryl-β-amino acid compounds of the invention can be synthesized using art recognized techniques or the procedures described herein. For example, β-heteroaryl-acrylic acid esters are accessible by reacting the corresponding heteroaryl aldehydes or heteroarylbromides via the Horner-Wadsworth-Emmons reaction or the Heck reaction, respectively (for reviews of the HWE reaction, see Wadsworth, Org. React. (1997) 25:73-253; Acc. Chem. Res. (1983) 16:411-417; Wadsworth J. Am. Chem. Soc. (1961) 83:1733; for reviews of the Heck reaction, see Heck, Palladium Reagents in Organic Syntheses; Academic Press: New York, 1985, pp. 179-321). The β-heteroaryl acrylic acid esters can also be treated (via a Michael addition) with secondary lithium amides to yield protected β-heteroaryl-β-amino acids (see, for example, March, Advanced Organic Chemistry; John Wiley & Sons: New York, 1992, pp. 795-797; J. G. Rico et al., J. Org. Chem. 58:27 7948-7951 (1993); and references cited therein). The protected β-amino acids can be deprotected and saponified to yield the β-heteroaryl-β-amino acids of the invention. β-Heteroaryl-β-amino acids can alternatively be prepared from the corresponding aldimines with subsequent Reformatsky reaction and deprotection. The synthetic methods of the invention are described in greater detail in the Examples.

In one embodiment, the compounds described herein do not include those mentioned in published PCT application WO 98/40055, incorporated herein by reference in its entirety.

The term “alkenyl” includes unsaturated aliphatic groups containing a carbon-carbon double bond, including straight-chain alkenyl groups, branched-chain alkenyl groups, cycloalkenyl (alicyclic) groups, alkenyl substituted cycloalkyl or cycloalkenyl groups, and cycloalkenyl substituted alkyl or alkenyl groups. The term alkenyl further includes alkenyl groups, which can further include oxygen, nitrogen, sulfur or phosphorous atoms replacing one or more carbons of the hydrocarbon backbone. In an embodiment, a straight chain or branched chain alkenyl group has 20 or fewer carbon atoms in its backbone (e.g., C₂-C₂₀ for straight chain, C₃-C₂₀ for branched chain).

The term “alkyl” includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone. In preferred embodiments, a straight chain or branched chain alkyl has 10 or fewer carbon atoms in its backbone (e.g., C₁-C₁₀ for straight chain, C₃-C₁₀ for branched chain), and more preferably 6 or fewer. Likewise, preferred cycloalkyls have from 4-7 carbon atoms in their ring structure, and more preferably have 5 or 6 carbons in the ring structure.

Moreover, the term alkyl includes both “unsubstituted alkyls” and “substituted alkyls,” the latter of which refers to alkyl moieties having substituents replacing one or more hydrogens on one or more carbons of the hydrocarbon backbone. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulflhydryl, alkylthio, arylthio, thiocarboxylate, sulfates, sulfonato, sulfamoyl, sulfonamido, nitro, trifluoromethyl, azido, heterocyclyl, alkylaryl, or an aromatic or heteroaromatic moiety. It will be understood by those skilled in the art that the moieties substituted on the hydrocarbon chain can themselves be substituted, if appropriate. Cycloalkyls can be further substituted, e.g., with the substituents described above. An “alkylaryl” moiety is an alkyl substituted with an aryl (e.g., phenylmethyl (i.e., benzyl)).

The term “aryl” includes aryl groups, including 5- and 6-membered single-ring aromatic groups that may include from zero to four heteroatoms, for example, phenyl, pyrrolyl, furanyl, thienyl, imidazolyl, benzoxazolyl, benzothiazolyl, triazolyl, tetrazolyl, pyrazolyl, pyridinyl, pyrazinyl, pyridazinyl, and pyrimidinyl, and the like. Aryl groups also include polycyclic fused aromatic groups such as naphthyl, quinolyl, indolyl, and the like. Those aryl groups having heteroatoms in the ring structure may also be referred to as “aryl heterocycles,” “heteroaryls” or “heteroaromatics”. The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The terms “alkenyl” and “alkynyl” include unsaturated aliphatic groups analogous in length and possible substitution to the alkyls described above, but that contain at least one double or triple bond, respectively. Examples of substituents of alkynyl groups include, for example alkyl, alkenyl (e.g., cycloalkenyl, e.g., cyclohexenyl), and aryl groups.

Unless the number of carbons is otherwise specified, “lower alkyl” as used herein means an alkyl group, as defined above, but having from one to three carbon atoms in its backbone structure. Likewise, “lower alkenyl” and “lower alkynyl” have similar chain lengths.

The terms “alkoxyalkyl,” “polyaminoalkyl,” and “thioalkoxyalkyl” include alkyl groups, as described above, which further include oxygen, nitrogen or sulfur atoms replacing one or more carbons of the hydrocarbon backbone, e.g., oxygen, nitrogen or sulfur atoms.

The term “polycyclyl” refers to two or more cyclic rings (e.g., cycloalkyls, cycloalkenyls, aryls or heterocyclyls) in which two or more carbons are common to two adjoining rings, e.g., the rings are “fused rings.” Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, azido, heterocyclyl, alkyl, alkylaryl, or an aromatic or heteroaromatic moiety.

The term “heteroatom” includes atoms of any element other than carbon or hydrogen. Preferred heteroatoms are nitrogen, oxygen, sulfur and phosphorus.

The term “alkylsulfinyl” include groups which have one or more sulfinyl (SO) linkages, typically 1 to about 5 or 6 sulfinyl linkages. Advantageous alkylsulfinyl groups include groups having 1 to about 12 carbon atoms, preferably from 1 to about 6 carbon atoms.

The term “alkylsulfonyl” includes groups which have one or more sulfonyl (SO₂) linkages, typically 1 to about 5 or 6 sulfonyl linkages. Advantageous alkylsulfonyl groups include groups having 1 to about 12 carbon atoms, preferably from 1 to about 6 carbon atoms.

The term “alkanoyl” includes groups having 1 to about 4 or 5 carbonyl groups. The term “aroyl” includes aryl groups, such as phenyl and other carbocyclic aryls, which have carbonyl substituents. The term “alkaroyl” includes aryl groups with alkylcarbonyl substituents, e.g., phenylacetyl.

It will be noted that the structure of some of the compounds of this invention includes asymmetric carbon atoms. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis. Furthermore, alkenes can include either the E- or Z-geometry, where appropriate.

The invention also pertains, at least in part, to novel compounds per se, e.g., anti-epileptogenic agents, described herein. Furthermore, the invention also pertains to pharmaceutical compositions comprising each of the chemical compounds described herein and packaged pharmaceutical compositions comprising any chemical compound described herein, packaged with directions relating to using the compounds to treat a nervous system disorder, e.g., an epileptogenic disorder, e.g., epilepsy.

In one embodiment, the invention provides a method for inhibiting epileptogenesis in a subject. The method includes the step of administering to a subject in need thereof an effective amount of a compound (e.g., an anti-epileptogenic agent of the invention, e.g., a β-heterocyclic-β-amino acid) which modulates a process in a pathway associated with epileptogenesis, such that epileptogenesis is inhibited in the subject.

As noted above, upregulation of excitatory coupling between neurons, mediated by N-methyl-D-aspartate (NMDA) receptors, and downregulation of inhibitory coupling between neurons, mediated by gamma-amino-butyric acid (GABA) receptors, have both been implicated in epileptogenesis. Other processes in pathways associated with epileptogenesis include release of nitric oxide (NO), a neurotransmitter implicated in epileptogenesis; release of calcium (Ca²⁺), which may mediate damage to neurons when released in excess; neurotoxicity due to excess zinc (Zn²⁺); neurotoxicity due to excess iron (Fe²⁺); and neurotoxicity due to oxidative cell damage. Accordingly, in preferred embodiments, an agent to be administered to a subject to inhibit epileptogenesis is capable of inhibiting one or more processes in at least one pathway associated with epileptogenesis. For example, an agent useful for inhibition of epileptogenesis can reduce the release of, or attenuate the epileptogenic effect of, NO in brain tissue; antagonize an NMDA receptor; augment endogenous GABA inhibition; reduce the release of, reduce the free concentration of (e.g., by chelation), or otherwise reduce the epileptogenic effect of cations including Ca²⁺, Zn²⁺, or Fe²⁺; inhibit oxidative cell damage; or the like. In certain preferred embodiments, an agent to be administered to a subject to inhibit epileptogenesis is capable of inhibiting at least two processes in at least one pathway associated with epileptogenesis.

In one preferred embodiment, the anti-epileptogenic agent antagonizes an NMDA receptor and augments endogenous GABA inhibition. In certain embodiments, the anti-epileptogenic agent is administered orally; preferably, after the step of oral administration, the anti-epileptogenic agent is transported to the nervous system of the subject by an active transport shuttle mechanism. A non-limiting example of an active transport shuttle is the β-amino acid transporter, which is capable of transporting β-amino acids across the blood-brain barrier (BBB).

The step of administering to a subject an anti-epileptogenic compound of the invention, e.g., a β-heterocyclic-β-amino acid or a compound of any Formula herein, can include administration to the subject of an anti-epileptogenic agent of the invention, an anti-epileptogenic agent in its active form, optionally in a pharmaceutically acceptable carrier. The step of administering to the subject can also include administering to the subject an agent which is metabolized to an anti-epileptogenic compound of the invention. For example, the methods of the invention include the use of prodrugs which are converted in vivo to the agents of the invention (see, e.g., R. B. Silverman, 1992, “The Organic Chemistry of Drug Design and Drug Action,” Academic Press, Chp. 8). Such prodrugs can be used to alter the biodistribution (e.g., to allow compounds which would not typically cross the blood-brain barrier to cross the blood-brain barrier) or the pharmacokinetics of the agent. For example, the anionic moiety, e.g., a carboxylate group, can be esterified, e.g., with an ethyl group or a fatty group, to yield a carboxylic acid ester. When the carboxylic acid ester is administered to a subject, the ester can be cleaved, enzymatically or non-enzymatically, to reveal the anionic moiety.

In an embodiment, an anti-epileptogenic agent of the invention may antagonize NMDA receptors by interacting with them, e.g., by binding to the glycine binding site. In another embodiment, the agent augments GABAergic inhibition by decreasing synaptic GABA uptake by glial cells. In certain other embodiments, the method further includes administering the agent in a pharmaceutically acceptable vehicle, e.g., such that the anti-epileptogenic agent is suitable, e.g., for oral administration.

In still another embodiment, the invention provides a method of treating (e.g., preventing, alleviating, modulating, etc.) convulsions (e.g., seizures, e.g., associated with epilepsy, trauma, etc.). The method includes the step of administering to a subject (e.g., a subject suffering from, or at risk of suffering from convulsions or a disorder characterized by convulsions or seizures) an effective amount of an anti-epileptogenic compound of the invention such that the convulsive disorder is treated. Examples of anti-epileptogenic agents of the invention include compounds such as β-heterocyclic-β-amino acids and compounds of any Formula herein.

In another embodiment, the invention provides a method for inhibiting both a convulsive condition and epileptogenesis in a subject. The method includes the step of administering to a subject in need thereof an effective amount of an agent which a) blocks sodium or calcium ion channels, or opens potassium or chloride ion channels; and b) has at least one activity selected from the group consisting of NMDA receptor antagonism; augmentation of endogenous GABA inhibition; calcium binding; iron binding; zinc binding; NO synthase inhibition; and antioxidant activity; such that epileptogenesis is inhibited in the subject.

Blockers of sodium or calcium ion channel activity are well known in the art and can be used as the A moiety in the compounds and methods of the present invention. Similarly, any compound which opens potassium or chloride ion channels can be used as the A moiety in the compounds and methods of the present invention. Antagonists of NMDA receptors and agonists of endogenous GABA inhibition are also known to one of skill in the art and can be used in the methods and compounds of the invention. For example, 2,3-quinoxalinediones are reported to have NMDA receptor antagonistic activity (see, e.g., U.S. Pat. No. 5,721,234). Exemplary calcium and zinc chelators include moieties known in the art for chelation of divalent cations, including (in addition to those mentioned supra) ethylenediaminetetraacetic acid (EDTA), ethylene glycol bis(beta-aminoethyl ether)-N,N,N′,N′-tetraacetic acid, and the like. Exemplary iron chelators include enterobactin, pyridoxal isonicotinyl hydrazones, N,N′-bis(2-hydroxybenzoyl)-ethylenediamine-N,N′-diacetic acid (HBED), 1-substituted-2-alkyl-3-hydroxy-4-pyridones, including 1-(2′-carboxyethyl)-2-methyl-3-hydroxy-4-pyridone, and other moieties known in the art to chelate iron. Compounds which inhibit NO synthase activity are known in the art and include, e.g., N γ-substituted arginine analogs (especially of the L configuration), including L-Nγ-nitro-arginine (a specific inhibitor of cerebral NO synthase), L-Nγ-amino-arginine, and L-Nγ-alkyl-arginines; or an ester (preferably the methyl ester) thereof. Exemplary antioxidants include ascorbic acid, tocopherols including alpha-tocopherol, and the like.

Anti-epileptogenic agents of the invention can be identified through screening assays. For example, the animal model of Phase 1 epileptogenesis described in Example 2, infra, can be employed to determine whether a particular compound has anti-epileptogenic activity against Phase 1 epileptogenesis. Chronic epileptogenesis can be modeled in rats (and candidate compounds screened with) the kindling assay described by Silver et al. (Ann. Neurol. (1991) 29:356). Similarly, compounds useful as anti-convulsants can be screened in conventional animal models, such as the mouse model described in Hotrod, R. W. et al., Eur. J. Pharmacol. (1979) 59:75-83. Compounds useful for, e.g., binding to or inhibition of receptors or enzymes can be screened according to conventional methods known to the ordinarily skilled artisan. For example, binding to the GABA uptake receptor can be quantified by the method of Ramsey et al. as modified by Schlewer (Schlewer, J., et al., J. Med. Chem. (1991) 34:2547). Binding to the glycine site on an NMDA receptor can be quantified, e.g., according to the method described in Kemp, A., et al., Proc. Natl. Acad. Sci. USA (1988) 85:6547. Effect on the voltage-gated Na⁺ channel can be evaluated in vitro by voltage clamp assay in rat hippocampal slices.

Assays suitable for screening candidate compounds for anticonvulsive or anti-epileptogenic activity in mice or rats are described in the Examples, infra.

In a further embodiment, the invention pertains, at least in part, to a method of diagnosing an epileptogenesis-associated condition in a subject. The method includes administering an anti-epileptogenic agent (e.g., a compound of any Formula herein), labeled with a detectable marker to the subject; and measuring increased binding of the compound to the NMDA receptors of the neurons of the subject's brain.

In yet another embodiment, the invention pertains, at least in part, to a method of diagnosing an epileptogenesis-associated state. The method includes administering an anti-epileptogenic agent (e.g., a compound of any Formula herein) labeled with a detectable marker to a subject; and measuring decreased binding of the compound to the GABA receptors of the neurons of the subject's brain.

In one embodiment, the invention pertains to pharmaceutical compositions, which include an effective amount of an anti-epileptogenic agent and a pharmaceutical acceptable carrier. The anti-epileptogenic agent may be a β-heterocyclic-β-amino acid (e.g., a β-heteroaromatic-β-amino acid), or a compound of Formula II: 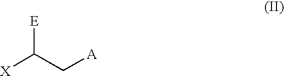
wherein:
   X is a heterocyclic moiety;
   E is a hydrogen bond donor;
   A is an hydrogen bond acceptor,
and pharmaceutically acceptable salts or esters, N-substituted analogs, C-substituted analogs, bioisosteres, and prodrugs thereof.

In another embodiment, the anti-epileptogenic agent in a pharmaceutical composition of the invention is of the Formula (IIa): 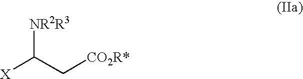
wherein:
R² and R³ are each independently hydrogen, alkyl, alkenyl, alkynyl, cycloalkyl, aryl, alkylaryl (e.g., benzyl and 1- or 2-phenethyl, i.e., α-methylbenzyl), alkylcarbonyl, arylcarbonyl (e.g., benzoyl), alkoxycarbonyl, or aryloxycarbonyl;
X is a heterocyclic moiety; and
R* is a substituted or unsubstituted alkyl moiety, a substituted or unsubstituted aryl moiety, a hydrogen, or a physiologically acceptable cation.

Other anti-epileptogenic agents which may be formulated into therapeutic compositions of the invention, include, but are not limited to, agents such as those compounds illustrated in the accompanying Examples and Table 1.

In a further embodiment, the effective amount is effective to treat an epileptogenesis-associated state in a subject. Examples of such states, include, but are not limited to, epilepsy, head trauma, pain, stroke, anxiety, schizophrenia, other psychoses, cerebral ischemia, Huntington's chorea, motor neuron disease, Alzheimer's disease, and dementia.

In another aspect, the present invention provides pharmaceutically acceptable compositions that comprise a therapeutically-effective amount of one or more of the agents described above, formulated together with one or more pharmaceutically acceptable carriers (additives) or diluents. The pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin; or (4) intravaginally or intrarectally, for example, as a pessary, cream or foam. In a preferred embodiment, the therapeutic compound is administered orally. The agents of the invention can be formulated as pharmaceutical compositions for administration to a subject, e.g., a mammal, including a human.

The agents of the invention are administered to subjects in a biologically compatible form suitable for pharmaceutical administration in vivo. By “biologically compatible form suitable for administration in vivo” is meant an administered agent wherein any toxic effects are outweighed by the therapeutic effects of the agent. The term “subject” is intended to include living organisms in which an immune response can be elicited, e.g., mammals. Examples of subjects include humans, dogs, cats, mice, rats, and transgenic species thereof. Administration of a therapeutically active amount of the therapeutic compositions of the present invention is defined as an amount effective, at dosages and for periods of time necessary to achieve the desired result. For example, a therapeutically active amount of an agent of the invention may vary according to factors such as the disease state, age, gender, and weight of the individual, and the ability of agent to elicit a desired response in the individual. Dosage regimes may be adjusted to provide the optimum therapeutic response. For example, several divided doses may be administered daily or the dose may be proportionally reduced as indicated by the exigencies of the therapeutic situation.

The active agent may be administered in a convenient manner such as by injection (subcutaneous, intravenous, etc.), oral administration, inhalation, transdermal application, or rectal administration. Depending on the route of administration, the active agent may be coated in a material to protect the agent from the action of enzymes, acids and other natural conditions which may inactivate the agent or to facilitate administration.

An agent of the invention can be administered to a subject in an appropriate carrier or diluent, co-administered with enzyme inhibitors or in an appropriate carrier such as liposomes. The term “pharmaceutically acceptable carrier” as used herein is intended to include diluents such as saline and aqueous buffer solutions. To administer an agent of the invention by other than parenteral administration, it may be necessary to coat the agent with, or co-administer the agent with a material to prevent its inactivation. Liposomes include water-in-oil-in-water emulsions as well as conventional liposomes (Strejan et al., (1984) J. Neuroimmunol 7:27). The active agent may also be administered parenterally or intraperitoneally. Dispersions can also be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The pharmaceutically acceptable carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating active agent in the required amount in an appropriate solvent with one or a combination of ingredients enumerated above, as required, followed by filter sterilization. Generally, dispersions are prepared by incorporating the active agent into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

When the active agent is suitably protected, as described above, the composition may be orally administered, for example, with an inert diluent or an edible carrier. As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active agent, use thereof in the therapeutic compositions is contemplated. Supplementary active agents can also be incorporated into the compositions.

It is especially advantageous to formulate parenteral compositions in dosage unit form for ease of administration and uniformity of dosage. “Dosage unit form,” as used herein refers to physically discrete units suited as unitary dosages for the mammalian subjects to be treated; each unit containing a predetermined quantity of active agent calculated to produce the desired therapeutic effect in association with the required pharmaceutical carrier. The specification for the dosage unit forms of the invention are dictated by and directly dependent on (a) the unique characteristics of the active agent and the particular therapeutic effect to be achieved, and (b) the limitations inherent in the art of compounding such an active agent for the therapeutic treatment of individuals.

### EXEMPLIFICATION OF THE INVENTION

### Example 1

### Synthesis of Some Compounds of the Invention

One skilled in the art will appreciate that the synthetic chemistry protocols described herein may be modified with no more than routine experimentation to arrive at analogous compounds which are therefore also within the scope of the present invention.

### N-[-1-methylbenzyl]-3-amino-3-(quinolin-2-yl)-propionic acid methyl ester

In a solution of acetonitrile, 2-quinolinecarboxaldehyde is treated with 3-(dimethoxy-phosphoryl)-acetic acid methyl ester in the presence of 1,8-diazabicyclo[5.4.0]undec-7-ene (DBU) and lithium chloride (Blanchette, et al., Tetrahedron Lett. 1984, 25, 2183-2186), to yield the β-quinolin-2-yl-acrylic acid ester. The acrylic acid ester is then treated with lithium 1-methylbenzyl-trimethylsilylamide in THF at −78° C. to yield the product via a Michael addition (J. G. Rico et al., J. Org. Chem. 58:27 7948-7951 (1993)), deprotection to follow.

### 3-Amino-3-(Benzo[d]-1,3-dioxolan-5-yl)propionic Acid

A mixture of benzo[d]-1,3-dioxolane-5-carboxaldehyde (3.04 g; 20.2 mmol), malonic acid (2.10 g; 20.1 mmol) and ammonium acetate (3.09 g; 40.1 mmol) in dry EtOH (40 mL) was refluxed for 6.5 hr. The resulting white solid was collected via filtration and triturated with EtOH (50 mL). A white powder was collected via filtration (0.78 g; 18%): mp 223-224° C.; R_{f} 0.24 (A); R_{f} 0.33 (B); νₘₐₓ (KBr): 3448, 2890, 1632, 1571, 1446, 1037 cm⁻¹; m/Z (ES): 210.1, 117.0. 76.0, 59.0; δ_{H} (D₂O, K₂CO₃, 400 MHz): 2.38 (1H, dd, J=14.5 and 7.1), 2.45 (1H, dd, J=14.5 and 7.7), 4.07 (1H, t, J=7.4), 5.81 (2H, d, J=1.2), 6.74 (2H, d, J=0.8), 6.80 (1H, s); δ_{C} (D₂O, K₂CO₃, 101 MHz): 46.9, 53.0, 101.3, 107.1, 108.7, 120.1, 138.7, 146.3, 147.4, 180.2; m/z calculated for C₁₀H₁₁NO₄: 210.0766 (MH⁺), found 210.0766 (MH⁺).

### 3-Amino-3-(Benzo[e]-1,4-dioxan-6-yl)propionic Acid

A mixture of benzo[e]-1,4-dioxane-6-carboxaldehyde (3.29 g; 20.0 mmol), malonic acid (2.08 g; 20.0 mmol) and ammonium acetate (3.10 g; 40.2 mmol) in dry EtOH (40 mL) was refluxed for 6.5 hr. The resulting white solid was collected via filtration and triturated with EtOH (50 mL). A white powder was collected via filtration (0.94 g; 13%): mp 222-223° C.; R_{f} 0.23 (A); R_{f} 0.37 (B); νₘₐₓ (KBr): 3443, 2875, 1631, 1564, 1071 cm⁻¹; m/z (ES): 224.1, 178.0, 117.0, 59.0; δ_{H} (D₂O, K₂CO₃, 400 MHz): 2.37 (1H, dd, J=15.4 and 6.9), 2.42 (1H, dd, J=14.5 and 7.8), 4.03 (1H, t, J=7.4), 4.13 (4H, s), 6.75 (2H, s), 6.77 (1H, s); δ_{C} (D₂O, K₂CO₃, 101 MHz): 46.9, 52.6, 64.8, 115.3, 117.5, 119.9, 138.2, 142.2, 143.0, 180.3; m/z calculated for C₁₁H₁₃NO₄: 224.0923 (MH⁺), found 224.0923 (MH⁺).

### 3-(Quinolin-2-yl)acrylic Acid Methyl Ester

To a stirred suspension of lithium chloride (1.02 g; 24.1 mmol) in dry MeCN (200 mL) under nitrogen at room temperature was added drop-wise at 15 min intervals trimethyl phosphonoacetate (4.39 g; 24.0 mmol) in dry MeCN (15 mL), DBU (3.05 g; 20.0 mmol) in dry MeCN (10 mL) and finally quinoline-2-carboxaldehyde (3.15 g; 20 mmol) in dry MeCN (30 mL). The reaction was allowed to stir at room temperature until completion, as determined by TLC. The reaction mixture was filtered and concentrated under reduced pressure. The resulting amber oil was dissolved in DCM (75 mL) and washed with distilled water (5×25 mL). The organic layer was dried over sodium sulfate and concentrated under reduced pressure to give a yellow solid. Purification by column chromatography on silica gel using EtOAc:DCM (1:19) as the eluent followed by recrystallization with EtOAc and hexanes gave a yellow crystalline solid (2.75 g; 65%): mp 85-86° C.; R_{f} 0.62 (D); R_{f} 0.20 (E); νₘₐₓ (KBr): 1733, 1282, 1121, 981 cm⁻¹; m/z (EI): 213.0, 182.0, 153.9; δ_{H} (CDCl₃, 200 MHz): 3.85 (3H, s), 7.00 (1H, d, J=15.8), 7.56 (1H, ddd, J=8.0, 6.8 and 1.2), 7.61 (1H, d, J=8.2), 7.74 (1H, ddd, J=8.2, 6.8 and 1.4), 7.83 (1H, dd, J=8.4 and 1.0), 7.90 (1H, d, J=15.8), 8.10 (1H, dq, J=8.4 and 1.0), 8.18 (1H, d, J=8.6); δ_{C} (CDCl₃, 126 MHz): 51.9, 120.2, 120.9, 123.1, 125.7, 126.4, 127.1, 127.2, 128.9, 129.9, 135.3, 136.6, 144.1, 147.2, 158.1, 173.1; m/z calculated for C₁₃H₁₁NO₂: 213.0790 (M⁺), found 213.0796 (M⁺).

### 3-(Quinolin-2-yl)acrylic Acid t-Butyl Ester

To a stirred suspension of lithium chloride (0.82 g; 19.3 mmol) in dry MeCN (140 mL) under nitrogen at room temperature was added drop-wise at 15 min intervals t-butyl P,P-dimethyl phosphonoacetate (4.31 g; 19.2 mmol) in dry MeCN (10 mL), DBU (2.43 g; 16.0 mmol) in dry MeCN (10 mL) and finally quinoline-2-carboxaldehyde (2.52 g; 16.0 mmol) in dry MeCN (30 mL). The reaction was allowed to stir at room temperature until completion, as determined by TLC. The reaction mixture was filtered and concentrated under reduced pressure. The resulting amber oil was dissolved in DCM (50 mL) and washed with distilled water (4×25 mL) and saturated sodium chloride solution (4×25 mL). The organic layer was dried over sodium sulfate and concentrated under reduced pressure to give a yellow solid. Purification by recrystallization with EtOAc and hexanes gave a yellow crystalline solid (2.54 g; 62%): mp 96-97° C.; R_{f} 0.53 (C); R_{f} 0.38 (E); νₘₐₓ (KBr): 3048, 1704, 1298, 1144, 992 cm⁻¹; m/z (EI): 255.1, 182.0, 153.8; δ_{H} (CDCl₃, 200 MHz): 1.56 (9H, s), 6.90 (1H, d, J=16.0), 7.56 (1H, dd, J=7.0 and 1.2), 7.62 (1H, d, J=8.6), 7.74 (1H, ddd, J=8.6, 6.8 and 1.6), 7.80 (1H, dd, J=8.2 and 1.0), 7.81 (1H, d, J=15.6), 8.12 (1H, d, J=8.6), 8.18 (1H, d, J=9.0); δ_{C} (CDCl₃, 126 MHz): 28.0, 80.6, 119.8, 125.6, 126.9, 127.3, 127.7, 129.5, 129.7, 136.4, 142.8, 148.0, 153.3, 165.5; m/z calculated for C₁₆H₁₇NO₂: 255.1260 (M⁺), found 255.1268 (M⁺).

### 3-(2-Chloroquinolin-3-yl)acrylic Acid Methyl Ester

To a stirred suspension of lithium chloride (1.02 g; 24.1 mmol) in dry MeCN (185 mL) under nitrogen at room temperature was added drop-wise at 15 min intervals trimethyl phosphonoacetate (4.39 g; 24.0 mmol) in dry MeCN (15 mL), DBU (3.05 g, 20.0 mmol) in dry MeCN (10 mL) and finally 2-chloroquinoline-3-carboxaldehyde (3.87 g; 20.1 mmol) in dry MeCN (40 mL). The reaction was allowed to stir at room temperature until completion, as determined by TLC. The reaction mixture was filtered and concentrated under reduced pressure. The resulting amber oil was dissolved in DCM (75 mL) and washed with distilled water (6×25 mL). The organic layer was dried over sodium sulfate and concentrated under reduced pressure to give a yellow solid. Purification by recrystallization with EtOAc and hexanes gave a yellow crystalline solid (2.52 g; 51%): mp 153-154° C.; R_{f} 0.65 (D); R_{f} 0.46 (E); νₘₐₓ (KBr): 1711, 1262, 1184, 980 cm⁻¹; m/z (EI): 247.0, 212.1, 153.1, 139.9, 127.1, 75.4; δ_{H} (CDCl₃, 200 MHz): 3.86 (3H, s), 6.57 (1H, dd, J=16.0 and 0.4), 7.60 (1H, ddd, J=8.2, 7.0 and 1.2), 7.78 (1H, ddd, J=8.4, 7.0 and 1.4), 7.86 (1H, dd, J=7.2 and 1.0), 8.02 (1H, dd, J=8.2 and 0.6), 8.14 (1H, dd, J=16.0 and 0.8), 8.84 (1H, s); δ_{H} (CDCl₃, 126 MHz): 53.4, 123.7, 129.1, 129.4, 129.8, 131.9, 133.0, 137.2, 137.5, 141.1, 149.3, 151.4, 167.8; m/z calculated for C₁₃H₁₀NO₂Cl: 247.0400 (M⁺), found 247.0406 (M⁺).

### 3-(Benzo[d]thiophen-3-yl)acrylic Acid Methyl Ester

To a stirred suspension of lithium chloride (1.02 g; 24.1 mmol) in dry MeCN (180 mL) under nitrogen at room temperature was added drop-wise at 15 min intervals trimethyl phophonoacetate (4.39 g; 24.0 mmol) in dry MeCN (15 mL), DBU (3.05 g; 20.0 mmol) in dry MeCN and finally benzo[d]thiophen-3-carboxaldehyde (3.24 g; 20.0 mmol) in dry MeCN (30 mL). The reaction was allowed to stir at room temperature until completion, as determined by TLC. The reaction mixture was filtered and concentrated under reduced pressure. The resulting yellow oil was dissolved in DCM (60 mL) and washed with distilled water (3×20 mL) and saturated sodium chloride solution (3×20 mL). The organic layer was dried over sodium sulfate and concentrated under reduced pressure to give a red oil. Purification by column chromatography on silica gel using DCM as the eluent followed by recrystallization with hexanes gave a pale yellow crystalline solid (2.63 g; 60%): mp 63-65° C.; R_{f} 0.68 (D); R_{f} 0.45 (F); νₘₐₓ (KBr): 1708, 1281, 1159, 972 cm⁻¹; m/z (EI): 218.0, 187.0, 159.1, 88.8; δ_{H} (CDCl₃, 200 MHz): 3.84 (3H, s), 6.54 (1H, d, J=15.8), 7.41 (1H, td, J=5.6 and 1.8), 7.47 (1H, td, J=5.6 and 1.6), 7.76 (1H, s), 7.88 (1H, dq, J=7.0 and 2.2), 7.98 (1H, dd, J=16.4 and 0.6), 8.02 (1H, dq, J=5.0 and 1.4); δ_{C} (CDCl₃, 101 MHz): 52.1, 118.6, 122.4, 123.3, 124.6, 125.4, 128.4, 131.9, 136.9, 137.4, 140.8, 167.9; m/z calculated for C₁₂H₁₀O₂S: 218.0402 (M⁺), found 218.0401 (M⁺).

### 3-(Benzo[d]furan-2-yl)acrylic Acid Methyl Ester

To a stirred suspension of lithium chloride (1.02 g; 24.1 mmol) in dry MeCN (200 mL) under nitrogen at room temperature was added drop-wise at 15 min intervals trimethyl phosphonoacetate (4.39; 24.0 mmol) in dry MeCN (15 mL), DBU (3.05 g; 20.0 mmol) in dry MeCN (10 mL) and finally benzo[b]furan-2-carboxaldehyde (2.92 g; 20.0 mmol) in dry MeCN (15 mL). The reaction was allowed to stir at room temperature until completion, as determined by TLC. The reaction mixture was filtered and concentrated under reduced pressure. The resulting yellow oil was dissolved in DCM (50 mL) and washed with distilled water (3×20 mL) and saturated sodium chloride solution (3×20 mL). The organic layer was dried over sodium sulfate and concentrated under reduced pressure to give an off-white solid. Purification by recrystallization with hexanes gave an off-white crystalline solid (3.98; 98%): mp 84-86° C.; R_{f} 0.60 (E); R_{f} 0.51 (C); νₘₐₓ (KBr): 3116, 1699, 1657, 1268, 1185, 956, 758 cm⁻¹; m/z (EI): 202.0, 171.0, 143.0, 130.9, 88.8; δ_{H} (CDCl₃, 200 MHz): 3.82 (3H, s), 6.58 (1H, d, J=15.8), 6.94 (1H, s), 7.23 (1H, td, J=7.2 and 1.2), 7.36 (1H, td, J=7.2 and 1.6), 7.48 (1H, dq, J=8.8 and 0.8), 7.56 (1H, d, J=16.0), 7.58 (1H, dq, J=7.8 and 0.8); δ_{C} (CDCl₃, 126 MHz): 51.6, 111.0, 111.2, 118.3, 121.6, 123.1, 126.3, 128.2, 131.3, 152.1, 155.4, 166.9; m/z calculated for C₁₂H₁₀O₃: 202.0630 (M⁺), found 202.0638 (M⁺).

### 3-(Benzo[d]furan-2-yl)acrylic Acid t-Butyl Ester

To a stirred suspension of lithium chloride (1.02 g; 24.1 mmol) in dry MeCN (175 mL) under nitrogen at room temperature was added drop-wise at 15 min intervals t-butyl P,P-dimethyl phosphonoacetate (5.38; 24.0 mmol) in dry MeCN (15 mL), DBU (3.05 g; 20.0 mmol) in dry MeCN (10 mL) and finally benzo[b]furan-2-carboxaldehyde (2.92 g; 20.0 mmol) in dry MeCN (40 mL). The reaction was allowed to stir at room temperature until completion, as determined by TLC. The reaction mixture was filtered and concentrated under reduced pressure. The resulting yellow oil was dissolved in DCM (60 mL) and washed with distilled water (3×25 mL) and saturated sodium chloride solution (3×25 mL). The organic layer was dried over sodium sulfate and concentrated under reduced pressure to give a yellow oil. Purification by column chromatography with EtOAc:hexanes (1:4) as the eluent gave a white powder (4.28 g; 89%): mp 56-57° C.; R_{f} 0.58 (C); R_{f} 0.82 (L); νₘₐₓ (KBr): 1694, 1635, 1295, 1161, 985 cm⁻¹; m/z (EI): 244.1, 188.0, 170.8, 131.0, 117.9, 114.8; δ_{H} (CDCl₃, 400 MHz): 1.56 (9H, s), 6.54 (1H, d, J=15.7), 6.90 (1H, s), 7.24 (1H, td, J=7.9 and 1.0), 7.35 (1H, td, J=7.2 and 1.3), 7.46 (1H, d, J=15.7), 7.47 (1H, dd, J=8.3 and 0.8), 7.58 (1H, dd, J=7.3 and 0.7); δ_{C} (CDCl₃, 101 MHz): 28.5, 81.0, 110.7, 111.7, 121.4, 122.0, 123.5, 126.5, 128.7, 130.6, 152.9, 155.8, 166.2; m/z calculated for C₁₅H₁₆O₃: 244.1099 (M⁺), found 244.1104 (M⁺).

### 3-(Benzo[e]-1,4-dioxan-6-yl)acrylic Acid Methyl Ester

To a stirred suspension of lithium chloride (1.02 g; 24.1 mmol) in dry MeCN (170 mL) under nitrogen at room temperature was added drop-wise at 15 min intervals trimethyl phophonoacetate (4.39 g; 24.0 mmol) in dry MeCN (15 mL), DBU (3.05 g, 20.0 mmol) in dry MeCN (15 mL) and finally benzo[e]-1,4-dioxane-6-carboxaldehyde (3.29 g; 20.0 mmol) in dry MeCN (40 mL). The reaction was allowed to stir at room temperature until completion, as determined by TLC. The reaction mixture was filtered and concentrated under reduced pressure. The resulting yellow oil was dissolved in DCM (50 mL) and washed with distilled water (4×20 mL) and saturated sodium chloride solution (3×20 mL). The organic layer was dried over sodium sulfate and concentrated under reduced pressure to give a yellow solid. Purification by recrystallization with EtOAc and hexanes gave a pale yellow crystalline solid (3.97 g; 90%): mp 66-68° C.; R_{f} 0.52 (E); R_{f} 0.30 (C); νₘₐₓ (KBr): 3013, 1708, 1693, 1281, 1172, 1152, 980, 804 cm⁻¹; m/z (EI): 220.0, 204.9, 189.0, 161.0, 150.9, 58.9; δ_{H} (CDCl₃, 200 MHz): 3.78 (3H, s), 4.27 (4H, s), 6.27 (1H, d, J=16.0), 6.85 (1H, dt, J=8.2 and 0.6), 7.00 (1H, ddd, J=8.8, 2.2, and 0.4), 7.04 (1H, d, J=0.6), 7.57 (1H, d, J=15.6); δ_{H} (CDCl₃, 126 MHz): 51.2, 64.0, 64.3, 115.6, 116.4, 117.4, 121.8, 127.8, 143.5, 144.2, 145.5, 167.3; m/z calculated for C₁₂H₁₂O₄: 220.0736 (M⁺), found: 220.0740 (M⁺).

### 3-(Benzo[d]dioxolan-5-yl)acrylic Acid t-Butyl Ester

To a stirred suspension of lithium chloride (1.02 g; 24.0 mmol) in dry MeCN (175 mL) under nitrogen at room temperature was added drop-wise at 15 min intervals t-butyl P,P-dimethyl phosphonoacetate (5.38 g; 24.0 mmol) in dry MeCN (15 mL), DBU (3.05 g; 20.0 mmol) in dry MeCN (10 mL) and finally benzo[d]dioxolane-5-carboxaldehyde (3.01 g; 20.0 mmol) in dry MeCN (40 mL). The reaction was allowed to stir at room temperature until completion, as determined by TLC. The reaction mixture was filtered and concentrated under reduced pressure. The resulting yellow oil was dissolved in DCM (60 mL) and washed with distilled water (3×25 mL) and saturated sodium chloride solution (3×25 mL). The organic layer was dried over sodium sulfate and concentrated under reduced pressure to give an off-white solid. Purification by recrystallization with MeOH gave a white crystalline solid (2.70 g; 54%): mp 82-83° C.; R_{f} 0.69 (D); R_{f} 0.63 (E); νₘₐₓ (KBr): 1699, 1635, 1248, 1145, 1100, 971 cm⁻¹; m/z (EI):248.2, 191.0, 175.0, 147.1, 145.0, 116.9, 89.0, 65.0; δ_{H} (CDCl₃, 400 MHz): 1.53 (9H, s), 5.99 (2H, s), 6.20 (1H, d, J=15.9), 6.79 (1H, d, J=8.0), 6.97 (1H, dd, J=8.0 and 1.5), 7.02 (1H, d, J=1.4), 7.49 (1H, d, J=15.9); δ_{C} (CDCl₃, 101 MHz): 28.5, 80.6, 101.8, 106.8, 108.9, 118.5, 124.4, 129.4, 143.5, 148.6, 149.6, 166.8; m/z calculated for C₁₄H₁₆O₄: 248.1049 (M⁺), found 248.1055 (M⁺).

### 3-(Indol-5-yl)acrylic Acid Methyl Ester

A mixture of 5-bromoindole (1.97 g, 10.0 mmol), methyl acrylate (1.08 g, 12.5 mmol), palladium acetate (24.9 mg, 0.1 mmol), tri(o-tolyl)phosphine (0.61 g, 2.0 mmol) and triethylamine (3.62 g, 35.8 mmol) was heated under argon in a heavy-walled Pyrex tube at 100° C. for 2 h. The cooled reaction mixture was diluted with DCM (60 mL) and distilled water (30 mL). The organic layer was washed with distilled water (3×25 mL), dried over sodium sulfate and concentrated under reduced pressure to give a yellow solid. Purification by recrystallization with EtOAc and hexanes gave a yellow powder (1.53 g; 76%): mp 138-140° C.; R_{f} 0.29 (C); R_{f} 0.51 (E); νₘₐₓ (KBr): 3316, 1694, 1284, 988 cm⁻¹; m/z (EI): 201.0, 170.0, 142.1, 116.1, 84.9; δ_{H} (CDCl₃, 200 MHz): 3.81 (3H, s), 6.42 (1H, d, J=15.4), 6.59 (1H, t, J=2.4), 7.24 (1H, d, J=3.0), 7.42 (2H, t, J=1.2), 7.81 (1H, s), 7.84 (1H, d, J=15.8), 8.34 (1H, bs); δ_{C} (CDCl₃, 126 MHz): 51.4, 103.1, 111.6, 114.2, 121.3, 122.3, 125.5, 126.1, 128.0, 137.1, 146.9, 168.3; m/z calculated for C₁₂H₁₁NO₂: 201.0790 (M⁺), found 201.0790 (M⁺).

### 3-(2-Methylindol-5-yl)acrylic Acid Methyl Ester

A mixture of 5-bromo-2-methylindole (2.10 g, 10.0 mmol), methyl acrylate (1.08 g, 12.5 mmol), palladium acetate (23.2 mg, 0.1 mmol), tri(o-tolyl)phosphine (61.3 g, 0.2 mmol) and triethylamine (3.62 g, 35.8 mmol) was heated under argon in a heavy-walled Pyrex tube at 100° C. for 3 h. The cooled reaction mixture was diluted with DCM (60 mL) and distilled water (30 mL). The organic layer was washed with distilled water (3×25 mL). The aqueous layer was extracted with DCM (25 mL). The combined organic layers were dried over sodium sulfate and concentrated under reduced pressure to give a pale yellow solid. Purification by recrystallization with EtOAc gave a pale yellow powder (1.57 g; 73%): mp 172-173° C.; R_{f} 0.35 (C); R_{f} 0.61 (E); νₘₐₓ (KBr): 3295, 1698, 1305, 1282, 1165, 975 cm⁻¹; m/z (EI): 215.1, 184.1, 156.1, 77.2; δ_{H} (CDCl₃, 400 MHz): 2.45 (3H, s), 3.82 (3H, s), 6.25 (1H, s), 6.42 (1H, d, J=16.0), 7.27 (1H, d, J=8.0), 7.35 (1H, d, J=8.0), 7.69 (1H, s), 7.85 (1H, d, J=16.0), 8.15 (1H, bs); δ_{C} (CDCl₃, 101 MHz): 14.0, 51.8, 101.5, 111.0, 114.5, 121.1, 121.4, 126.5, 129.7, 136.7, 137.7, 147.4, 168.6; m/z calculated for C₁₃H₁₃NO₂: 215.0946 (M⁺), found 215.0945 (M⁺).

### N-(t-Butyldimethyl)-3-(2-Methylindol-5-yl)acrylic Acid t-Butyl Ester

A mixture of N-(t-butyldimethyl)-5-bromo-2-methylindole (1.30 g, 4.0 mmol), t-butyl acrylate (0.64 g, 5.0 mmol), palladium acetate (25.6 mg, 0.1 mmol), tri(o-tolyl)phosphine (63.5 g, 0.2 mmol) and triethylamine (1.45 g, 14.3 mmol) was heated under argon in a heavy-walled Pyrex tube at 100° C. for 3 h. The cooled reaction mixture was diluted with DCM (60 mL) and distilled water (30 mL). The organic layer was washed with distilled water (3×25 mL). The aqueous layer was extracted with DCM (25 mL). The combined organic layers were dried over sodium sulfate and concentrated under reduced pressure to give a pale yellow oil. Purification by column chromatography using EtOAc:hexanes (1:9) as the eluent followed by recrystallization with hexanes gave a white powder (0.94 g; 63%): mp 102-103° C.; R_{f} 0.40 (H); R_{f} 0.33 (M); νₘₐₓ (KBr): 2951, 1708, 1631, 1302, 1271, 1150, 990 cm⁻¹; m/z (EI): 371.4, 315.3, 298.3, 258.2, 184.0, 155.9, 129.1, 115.0; δ_{H} (CDCl₃, 400 MHz): 0.65 (6H, s), 0.98 (9H, s), 1.56 (9H, s), 2.49 (3H, d, J=0.5), 6.34 (1H, d, J=15.9), 6.35 (1H, s), 7.27 (1H, dd, J=6.5 and 1.8), 7.47 (1H, d, J=8.7), 7.63 (1H, d, J=1.6), 7.71 (1H, d, J=15.9); δ_{C} (CDCl₃, 101 MHz): −0.2, 17.8, 20.9, 27.0, 28.6, 80.2, 106.9, 114.7, 117.2, 120.4, 120.5, 126.8, 132.0, 143.6, 144.3, 145.6, 167.4; m/z calculated for C₂₂H₃₃NO₂Si: 371.2281 (M⁺), found 371.2297 (M⁺).

### 3-(Quinolin-3-yl)acrylic Acid Methyl Ester

A mixture of 3-bromoquinoline (2.08 g, 10.0 mmol), methyl acrylate (1.08 g, 12.5 mmol), palladium acetate (23.6 mg, 0.1 mmol), tri(o-tolyl)phosphine (0.122 g, 0.4 mmol) and triethylamine (3.62 g, 35.8 mmol) was heated under argon in a heavy-walled Pyrex tube at 100° C. for 6 h. The cooled reaction mixture was diluted with DCM (60 mL) and distilled water (30 mL). The organic layer was washed with distilled water (3×25 mL). The aqueous layer was extracted with DCM (25 mL). The combined organic layers were dried over sodium sulfate and concentrated under reduced pressure to give a pale yellow solid. Purification by recrystallization with EtOAc and hexanes gave an off-white crystalline solid (1.82 g; 85%): mp 124-125° C.; R_{f} 0.19 (C); R_{f} 0.10 (E); νₘₐₓ (KBr): 1716, 1635, 1263, 1174, 983 cm⁻¹; m/z (EI): 213.0, 182.3, 154.2, 128.5; δ_{H} (CDCl₃, 200 MHz): 3.84 (3H, s), 6.67 (1H, d, J=16.2), 7.60 (1H, ddd, J=8.2, 7.2 and 1.4), 7.77 (1H, ddd, J=8.4, 7.0 and 1.6), 7.84 (1H, d, J=16.2), 7.86 (1H, dd, J=7.0 and 1.4), 8.14 (1H, d, J=8.6), 8.26 (1H, d, J=2.2), 9.09 (1H, d, J=2.2); δ_{C} (CDCl₃, 126 MHz): 51.7, 119.6, 127.2, 127.3, 127.4, 128.1, 129.2, 130.4, 135.3, 141.2, 148.4, 149.0, 166.7; m/z calculated for C₁₃H₁₁O₂N: 213.0790 (M⁺), found 213.0790 (M⁺).

### 3-(Quinolin-3-yl)acrylic Acid t-Butyl Ester

A mixture of 3-bromoquinoline (2.08 g, 10.0 mmol), t-butyl acrylate (1.60 g, 12.5 mmol), palladium acetate (25.1 mg, 0.1 mmol), tri(o-tolyl)phosphine (0.130 g, 0.4 mmol) and triethylamine (3.62 g, 35.8 mmol) was heated under argon in a heavy-walled Pyrex tube at 100° C. for 6 h. The cooled reaction mixture was diluted with DCM (60 mL) and distilled water (30 mL). The organic layer was washed with distilled water (3×25 mL). The aqueous layer was extracted with DCM (25 mL). The combined organic layers were dried over sodium sulfate and concentrated under reduced pressure to give a yellow solid. Purification by recrystallization with EtOAc and hexanes gave a pale yellow crystalline solid (2.43 g; 95%): mp 128-129° C.; R_{f} 0.32 (D); R_{f} 0.24 (L); νₘₐₓ (KBr): 1694, 1635, 1295, 1161, 984 cm⁻¹; m/z (EI): 255.0, 198.8, 181.8, 169.9, 154.1, 126.5; δ_{H} (CDCl₃, 200 MHz): 1.55 (9H, s), 6.58 (1H, d, J=16.4), 7.56 (1H, td, J=8.0 and 1.2), 7.72 (1H, d, J=16.4), 7.73 (1H, td, J=8.2 and 1.4), 7.82 (1H, dd, J=8.2 and 1.2), 8.20 (1H, d, J=2.2), 9.05 (1H, d, J=2.2); δ_{C} (CDCl₃, 101 MHz): 28.5, 81.3, 122.5, 127.7, 127.9, 128.0, 128.6, 29.6, 130.8, 135.7, 140.4, 148.6, 149.5, 166.0; m/z calculated for C₁₆H₁₇O₂N, 255.1259 (M⁺), found 255.1251 (M⁺).

### 3-(Isoquinolin-4-yl)acrylic Acid Methyl Ester

A mixture of 4-bromoisoquinoline (2.08 g, 10.0 mmol), methyl acrylate (1.08 g, 12.5 mmol), palladium acetate (24.2 mg, 0.1 mmol), tri(o-tolyl)phosphine (1.22 g, 4.0 mmol) and triethylamine (3.62 g, 35.8 mmol) was heated under argon in a heavy-walled Pyrex tube at 100° C. for 46 h. The cooled reaction mixture was diluted with DCM (60 mL) and distilled water (30 mL). The organic layer was washed with distilled water (4×25 mL). The aqueous layer was extracted with DCM (25 mL). The combined organic layers were dried over sodium sulfate and concentrated under reduced pressure to give a yellow solid. Purification by recrystallization with EtOAc and hexanes gave a yellow powder (1.41 g; 66%): mp 79-80° C.; R_{f} 0.10 (C); R_{f} 0.07 (E); νₘₐₓ (KBr): 1716, 1631, 1318, 1176, 976 cm⁻¹; m/z (EI): 213.0, 181.9, 154.0, 128.1; δ_{H} (CDCl₃, 200 MHz): 3.87 (3H, s), 6.61 (1H, d, J=15.8), 7.71 (1H, ddd, J=8.4, 6.8 and 1.2), 7.85 (1H, ddd, J=8.0, 6.8 and 1.2), 8.07 (1H, d, J=8.2), 8.17 (1H, d, J=7.6), 8.36 (1H, d, J=15.8), 8.74 (1H, s), 9.29 (1H, s); δ_{C} (CDCl₃, 126 MHz): 51.7, 121.7, 122.4, 125.4, 127.5, 127.9, 128.1, 131.0, 133.4, 138.7, 141.4, 153.8, 166.6; m/z calculated for C₁₃H₁₁NO₂: 213.0790 (M⁺), found 213.0786 (M⁺).

### 3-(Isoquinolin-4-yl)acrylic Acid t-Butyl Ester

A mixture of 4-bromoisoquinoline (2.08 g, 10.0 mmol), t-butyl acrylate (1.60 g, 12.5 mmol), palladium acetate (25.7 mg, 0.1 mmol), tri(o-tolyl)phosphine (0.132 g, 0.4 mmol) and triethylamine (3.62 g, 35.8 mmol) was heated under argon in a heavy-walled Pyrex tube at 100° C. for 46 h. The cooled reaction mixture was diluted with DCM (60 mL) and distilled water (30 mL). The organic layer was washed with distilled water (4×25 mL). The aqueous layer was extracted with DCM (25 mL). The combined organic layers were dried over sodium sulfate and concentrated under reduced pressure to give a yellow oil. Purification by column chromatography with EtOAc:DCM (1:9) as the eluent followed by recrystallization with hexanes gave a pale yellow crystalline solid (1.95 g; 76%): mp 81-83° C.; R_{f} 0.19 (C); R_{f} 0.22 (L); νₘₐₓ (KBr): 1702, 1627, 1318, 1150, 970 cm⁻¹; m/z (EI): 255.2, 199.1, 182.0, 154.0, 126.9, 76.8; δ_{H} (CDCl₃, 400 MHz): 1.57 (9H, s), 6.51 (1H, d, J=15.9), 7.64 (1H, t, J=7.3), 7.77 (1H, td, J=7.0 and 1.1), 7.99 (1H, d, J=8.2), 8.12 (1H, d, J=8.5), 8.24 (1H, d, J=15.9), 8.72 (1H, s), 9.21 (1H, s); δ_{C} (CDCl₃, 101 MHz): 28.5, 81.3, 123.0, 124.7, 126.3, 127.9, 128.4, 131.5, 134.0, 137.8, 141.6, 153.9, 166.0; m/z calculated for C₁₃H₁₁NO₂: 255.1259 (M⁺), found 255.1266 (M⁺).

### 3-(Thiophen-2-yl)acrylic Acid Methyl Ester

To a stirred solution of 3-(thiophen-2-yl)acrylic acid (3.08 g: 20.0 mmol) in dry MeOH (75 mL) at 0° C. under nitrogen was added drop-wise thionyl chloride (4.89 g; 40.0 mmol). The resulting mixture was allowed to warm to room temperature and refluxed until completion, as determined by TLC. To the reaction mixture was added activated carbon. The resulting mixture was filtered and concentrated under reduced pressure to give a tan solid (2.54 g; 76%): mp 52-54° C.; R_{f} 0.88 (A); R_{f} 0.53 (C); νₘₐₓ (KBr): 1708, 1306, 1164, 986, 703 cm⁻¹; m/z (EI): 168.0, 137.0, 108.7, 83.1; δ_{H} (CDCl₃, 200 MHz): 3.75 (3H, s), 6.21 (1H, d, J=15.8), 7.01 (1H, t, J=4.4), 7.21 (1H, d, J=3.2), 7.33 (1H, d, J=5.0), 7.76 (1H, d, J=15.4); δ_{C} (CDCl₃, 126 MHz): 51.4, 116.4, 127.9, 128.2, 130.7, 137.0, 139.3, 167.0; m/z calculated for C₈H₈O₂S: 168.0245 (M⁺), found 168.0246 (M⁺).

### N,N-(Dibenzyl)-3-Amino-3-(Thiophen-2-yl)propionic Acid Methyl Ester

To a stirred solution of dibenzylamine (0.789 g; 4.00 mmol) in dry THF (30 mL) at 0° C. under nitrogen was added drop-wise n-butyl lithium (1.6 M in hexanes; 2.5 mL; 4.0 mmol). The resulting red solution was stirred at 0° C. for 15 min and cooled to −78° C. 3-(thiophen-2-yl)acrylic acid methyl ester (0.340 g, 2.02 mmol) in dry THF (8 mL) was added drop-wise at −78° C. and stirred for 15 min before quenching with saturated ammonium chloride solution (4 mL). The reaction mixture was allowed to warm to room temperature and poured into saturated sodium chloride solution (50 mL). The aqueous layer was separated and extracted with diethyl ether (2×40 mL). The combined organic layers were washed with saturated sodium chloride solution (2×10 mL), dried over sodium sulfate and concentrated under reduced pressure to give a pale yellow oil. Purification by column chromatography on silica gel with Et₂O:hexanes (1:4) as the eluent gave white crystals (0.378 g; 51%): mp 88-90° C.; R_{f} 0.56 (C); R_{f} 0.29 (G); νₘₐₓ (KBr): 1739, 1609, 1294, 1257, 1116, 1021, 697 cm⁻¹; m/z (CI): 366.2, 292.1, 198.0, 168.9, 90.9, 82.8; δ_{H} (CDCl₃, 200 MHz): 2.78 (1H, dd, J=14.8 and 7.0), 3.07 (1H, dd, J=13.1 and 8.0), 3.36 (2H, d, J=13.6), 3.64 (3H, s), 3.71 (2H, d, J=13.8), 4.54 (1H, t, J=7.2), 6.90 (1H, dq, J=3.4 and 1.2), 7.01 (1H, ddd, J=5.0,3.4 and 0.8), 7.18-7.41(11H, m); δ_{C} (CDCl₃, 126 MHz): 37.6, 51.5, 53.7, 54.8, 124.5, 125.4, 126.4, 127.0, 128.1, 128.9, 139.2, 142.0, 171.5; m/z calculated for C₂₂H₂₃NO₂S: 365.1450 (M⁺), found 365.1451 (M⁺).

### N,N-(Dibenzyl)-3-Amino-3-(Quinolin-2-yl)propionic Acid Methyl Ester and N,N,N′,N′-(Tetrabenzyl)-3-Amino-3-(Quinolin-2-yl)-Propionoamide

To a stirred solution of dibenzylamine (3.95 g; 20.0 mmol) in dry THF (150 mL) at 0° C. under nitrogen was added drop-wise n-butyl lithium (1.6 M in hexanes; 12.5 mL; 20.0 mmol). The resulting red solution was stirred at 0° C. for 15 min and cooled to −78° C. 3-(Quinolin-2-yl)acrylic acid methyl ester (2.13 g; 10.0 mmol) in dry THF (30 mL) was added drop-wise at −78° C. and stirred for 15 min before quenching with saturated ammonium chloride solution (20 mL). The reaction mixture was allowed to warm to room temperature and poured into saturated sodium chloride solution (50 mL). The aqueous layer was separated and extracted with diethyl ether (2×25 mL). The combined organic layers were washed with saturated sodium chloride solution (3×40 mL), dried over sodium sulfate and concentrated under reduced pressure to give an amber oil. Purification by column chromatography on silica gel with Et₂O: hexanes (1:2) as the eluent followed by purification by recrystallization with Et₂O and hexanes gave two products. A yellow crystalline solid (Ester: 0.64 g; 16%): mp 101-102° C.; R_{f} 0.56 (E); R_{f} 0.30 (C); νₘₐₓ (KBr): 3058, 1728, 1296, 1218 cm⁻¹; m/z (CI): 411.3, 215.0, 155.9, 91.0; δ_{H} (CDCl₃, 200 MHz): 3.08 (1H, dd, J=16.0 and 4.0), 3.42 (1H, dd, J=15.4 and 9.2), 3.64 (4H, s), 3.66 (3H, s), 4.63 (1H, dd, J=8.4 and 4.4), 7.20-7.40 (10H, m), 7.50 (1H, t, J=7.0), 7.57 (1H, d, J=8.6), 7.67 (1H, td, J=7.0 and 1.6), 7.79 (1H, d, J=8.2), 8.03 (1H, d, J=8.4), 8.12 (1H, d, J=8.4); δ_{C} (CDCl₃, 126 MHz): 31.0, 51.5, 59.8, 121.9, 126.1, 127.0, 127.3, 127.4, 128.3, 128.9, 129.0, 129.5, 135.7, 139.6, 147.1, 160.1, 173.3; m/z calculated for C₂₇H₂₆N₂O₂: 411.2073 (MH⁺), found 411.2080 (MH⁺). A white crystalline solid (Amide: 1.47 g; 26%): mp 120-123° C.; R_{f} 0.62 (E); R_{f} 0.16 (H); νₘₐₓ (KBr): 3059, 1620, 1235 cm⁻¹; m/z (CI): 576.2, 379.2, 198.0, 183.9, 155.9, 90.9; δ_{H} (CDCl₃, 200 MHz): 2.78 (1H, d, J=15.6), 3.53 (2H, d, J=13.6), 3.70 (2H, d, J=13.4), 3.86 (1H, t, J=11.4), 4.10 (1H, d, J=15.0), 4.51 (1H, d, J=17.2), 4.99 (2H, t, J=14.8), 5.34 (1H, d, J=17.0), 6.93 (4H, s), 7.05-7.63 (18H, m), 7.91 (1H, d, J=8.2), 8.13 (1H, d, J=8.6); δ_{C} (CDCl₃, 126 MHz): 28.2, 48.3, 50.6, 54.4, 61.4, 122.9, 126.0, 126.8, 126.9, 127.0, 127.5, 127.6, 127.7, 128.3, 128.8, 129.4, 135.8, 137.3, 137.6, 139.8, 146.9, 161.0, 173.0; m/z calculated for C₄₀H₃₇N₃O: 576.3015 (MH⁺), found 576.2987 (MH⁺).

### N-(α-Methylbenzyl)-3-Amino-3-(Quinolin-2-yl)propionic Acid Methyl Ester

To a stirred solution of α-methylbenzylamine (2.43 g; 20.0 mmol) and triethylamine (2.86 g; 28.3 mmol) in dry THF (30 mL) at room temperature under argon was added drop-wise trimethylsilyl chloride (2.39 g; 23.6 mmol). The mixture was allowed to stir at room temperature for 1 h after which triethylamine hydrochloride was removed via filtration under a blanket of argon. The resulting clear silylamine, in dry THF, was cooled to −78° C. and n-butyl lithium (1.6 M in hexanes; 9.4 mL; 15.0 mmol) was added drop-wise and the mixture stirred for 15 min. To this solution was added drop-wise 3-(quinolin-2-yl)acrylic acid methyl ester (2.13 g; 10.0 mmol) in dry THF (5 mL). The resulting mixture was stirred at −78° C. for 15 min before quenching with saturated ammonium chloride solution (7.2 mL). The reaction mixture was allowed to warm to room temperature and extracted with Et₂O (3×25 mL). The combined organic layers were concentrated under reduced pressure, after which 1 N hydrochloric acid (15 mL) was added. The resulting mixture was washed with Et₂O (3×25 mL) and the organic layers discarded. The aqueous layer was basified with solid potassium carbonate and extracted with DCM (4×25 mL). The combined organic layers were dried over sodium sulfate and concentrated under reduced pressure to give a red oil. Purification by column chromatography using EtOAc:hexanes (1:2) as the eluent gave an amber oil (2.32 g; 70%): R_{f} 0.31 (C); R_{f} 0.48 (K); νₘₐₓ (nujol): 3449, 3332, 1953, 1736, 1264, 1166, 833, 759 cm⁻¹; m/z (EI): 335.2, 319.2, 303.2, 215.0, 181.9, 156.0, 127.8, 104.9, 76.9; δ_{H} (CDCl₃, 400 MHz): 1.43 (3H, d, J=6.5), 2.95 (2H, d, J=6.3 and 2.6), 3.65 (3H, s), 3.84 (1H, q, J=6.5), 4.43 (1H, t, J=6.6), 7.18-7.32 (6H, m), 7.41 (1H, d, J=8.4), 7.51 (1H, t J=7.1), 7.69 (1H, t, J=7.0), 7.78 (1H, d, J=7.1), 8.05 (2H, d, J=8.3); & (CDCl₃, 101 MHz): 23.7, 40.4, 51.9, 56.0, 58.6, 120.8, 126.4, 127.1, 127.1, 127.2, 127.7, 127.8, 128.6, 128.6, 129.6, 136.5, 146.0, 148.0, 162.8, 172.8; m/z calculated for C₂₁H₂₂N₂O₂: 335.1760 (MH⁺), found 335.1766 (MH⁺).

### N-(α-Methylbenzyl)-3-Amino-3-(Quinolin-2-yl)propionic Acid t-Butyl Ester

To a stirred solution of α-methylbenzylamine (0.728 g; 6.00 mmol) and triethylamine (0.857 g; 8.57 mmol) in dry THF (10 mL) at room temperature under argon was added drop-wise trimethylsilyl chloride (0.717 g; 7.07 mmol). The mixture was allowed to stir at room temperature for 1 h after which triethylamine hydrochloride was removed via filtration under a blanket of argon. The resulting clear silylamine, in dry THF, was cooled to −78° C. and n-butyl lithium (1.6 M in hexanes; 2.82 mL; 4.50 mmol) was added drop-wise and the mixture stirred for 15 min. To this solution was added drop-wise 3-(quinolin-2-yl)acrylic acid t-butyl ester (0.774 g; 3.03 mmol) in dry THF (2 mL). The resulting mixture was stirred at −78° C. for 15 min before quenching with saturated ammonium chloride solution (2.5 mL). The reaction mixture was allowed to warm to room temperature and extracted with Et₂O (3×25 mL). The combined organic layers were concentrated under reduced pressure, after which 1 N hydrochloric acid (10 mL) was added. The resulting mixture was washed with Et₂O (3×25 mL) and the organic layers discarded. The aqueous layer was basified with solid potassium carbonate and extracted with DCM (4×25 mL). The combined organic layers were dried over sodium sulfate and concentrated under reduced pressure to give a red oil. Purification by column chromatography using EtOAc:hexanes (1:2) as the eluent gave an amber oil (0.58 g; 50%): R_{f} 0.43 (C); R_{f} 0.66 (D); νₘₐₓ (nujol): 3431, 3331, 1952, 1727, 1259, 1153, 835, 758 cm⁻¹; m/z (EI): 377.3, 321.3, 257.2, 201.1, 127.9, 105.2, 156.1; δ_{H} (CDCl₃, 200 MHz): 1.30 (1H, d, J=6.6), 1.35 (3H, s), 1.38 (6H, s), 1.43 (2H, d, J=6.6), 2.70 (0.7H, d, J=7.0), 2.81 (1.3H, d, J=6.8), 3.52 (0.3H, q, J=6.0), 3.81 (0.7H, q, J=6.6), 4.10 (0.3H, t, J=7.2), 4.40 (0.7H, t, J=7.0), 7.25-7.38 (5H, m), 7.39 (1H, d, J=8.8), 7.50 (1H, m), 7.68 (1H, m), 7.78 (1H, dd, J=8.4 and 1.8), 8.03 (1H, d, J=8.4); δ_{C} (CDCl₃, 126 MHz): 29.5, 43.2, 57.2, 60.2, 121.7, 122.3, 127.5, 128.3, 128.5, 128.8, 128.9, 129.7, 129.8, 130.6, 130.7, 137.7, 149.1, 172.2; m/z calculated for C₂₄H₂₈N₂O₂: 377.2229 (MH⁺), found 377.2235 (MH⁺).

### N-(α-Methylbenzyl)-3-Amino-3-(Quinolin-2-yl)propionic Acid

To a stirred solution of N-(α-methylbenzyl)-3-amino-3-(quinolin-2-yl)propionic acid t-butyl ester (0.72 g; 1.9 mmol) in DCM (6 mL) was added drop-wise trifluoroacetic acid (5 mL). The reaction mixture was allowed to stir at room temperature overnight after which it was concentrated under reduced pressure to give a brown solid. The solid was dissolved in EtOAc (50 mL) and the resulting solution was washed with saturated sodium bicarbonate solution (3×10 mL), dried over sodium sulfate and concentrated under reduced pressure to give a rusty coloured solid. Purification by column chromatography using chloroform:MeOH (4:1) as the eluent gave a tan crystalline solid (0.50 g; 83%): mp 84° C. (decomposition); R_{f} 0.68 (B); R_{f} 0.34 (N); νₘₐₓ (nujol): 3443, 1686, 1602, 1421, 1132, 833, 761, 701 cm⁻¹; m/z (FAB): 321.3, 156.1, 128.1, 105.0; δ_{H} (CDCl₃, 500 MHz): 1.72 (3H, d, J=6.5), 2.96 (2H, s), 4.33 (1H, d, J=6.4), 4.87 (1H, s), 7.10 (3H, s), 7.29 (3H, d, J=5.7), 7.53 (1H, t, J=7.4), 7.69 (1H, t, J=7.6), 7.74 (1H, d, J=8.0), 7.97 (1H, d, J=8.3), 8.02 (1H, d, J=8.3); δ_{C} (CDCl₃, 126 MHz): 19.8, 39.9, 58.9, 59.1, 115.6, 118.5, 119.8, 127.5, 127.8, 127.9, 128.0, 129.1, 129.2, 129.6, 130.6, 138.4, 146.8, 155.4; m/z calculated for C₂₀H₂₀N₂O₂: 321.1602 (MH⁺), found 321.1603 (MH⁺).

### 3-Amino-3-(Quinolin-2-yl)propionic Acid t-Butyl Ester

A solution of N-(α-methylbenzyl)-3-amino-3-(quinolin-2-yl)propionic acid t-butyl ester (0.57 g; 1.5 mmol) in 1,4-cyclohexadiene (1.4 mL) and glacial acetic acid (5.5 mL) was treated with 10% palladium on carbon (0.437 g). This mixture was allowed to stir at 75° C. under argon until completion, as determined by TLC. The reaction mixture was then allowed to cool to room temperature and filtered through Celite®. The filtrate was concentrated under reduced pressure to give a yellow oil. Trituration with Et₂O (25 mL) gave an off-white solid (36.2 mg; 9%): mp 181-183° C.; R_{f} 0.45 (A); R_{f} 0.70 (B); νₘₐₓ (KBr): 3438, 1727, 1598, 1272, 1157 cm⁻¹; m/z (EI): 216.0, 199.0, 182.0, 171.0, 156.9, 129.0, 101.9, 89.1; δ_{H} (CDCl₃, 400 MHz): 1.30 (9H, s), 3.29 (2H, d, J=2.8), 5.16 (1H, s), 7.55 (2H, q, J=7.5), 7.68 (1H, t, J=7.3), 7.78 (1H, d, J=8.0), 8.06 (1H, d, J=8.4), 8.13 (1H, d, J=8.3); δ_{C} (CDCl₃, 101 MHz): 28.2, 39.0, 52.0, 82.5, 119.8, 127.5, 127.9, 128.0, 129.6, 130.4, 138.0, 147.1, 154.6, 169.4.

### N-(α-Methylbenzyl)-3-Amino-3-(Quinolin-3-yl)propionic Acid Methyl Ester

To a stirred solution of α-methylbenzylamine (1.69 g; 14.0 mmol) and triethylamine (2.03 g; 20.0 mmol) in dry THF (30 mL) at room temperature under argon was added drop-wise trimethylsilyl chloride (1.79 g; 16.5 mmol). The mixture was allowed to stir at room temperature for 1 h after which triethylamine hydrochloride was removed via filtration under a blanket of argon. The resulting clear silylamine, in dry THF, was cooled to −78° C. and n-butyl lithium (1.6 M in Hexanes; 6.56 mL; 10.5 mmol) was added drop-wise and the mixture stirred for 15 min. To this solution was added drop-wise 3-(quinolin-3-yl)acrylic acid methyl ester (1.50 g; 7.05 mmol) in a mixture of dry THF (20 mL) and toluene (1 mL). The resulting mixture was stirred at −78° C. for 15 min before quenching with saturated ammonium chloride solution (5 mL). The reaction mixture was allowed to warm to room temperature and extracted with Et₂O (3×25 mL). The combined organic layers were concentrated under reduced pressure, after which 1 N hydrochloric acid (10 mL) was added. The resulting mixture was washed with Et₂O (3×25 mL) and the organic layers discarded. The aqueous layer was basified with solid potassium carbonate and extracted with DCM (4×25 mL). The combined organic layers were dried over sodium sulfate and concentrated under reduced pressure to give a red oil. Purification by column chromatography using gradient elution with EtOAc:hexanes (1:2, 2:1 and 3:1) gave an amber oil (1.01 g; 43%): R_{f} 0.08 (C); R_{f} 0.34 (D); νₘₐₓ (nujol): 3449, 3327, 1956, 1735, 1266, 1168, 788 cm⁻¹; m/z (EI): 335.3, 319.3, 261.2, 229.1, 214.0, 1556.9, 104.9; δ_{H} (CDCl₃, 400 MHz): 1.42 (3H, d, J=6.0), 2.85 (2H, qd, J=16.0 and 8.0), 3.60 (3H, s), 3.75 (1H, q, J=6.0), 4.40 (1H, t, J=6.0), 7.11-7.19 (1H, m), 7.23 (5H, d, J=6.0), 7.52 (1H, t, J=8.0), 7.68 (1H, t, J=8.0), 7.76 (1H, d, J=8.0), 8.03 (1H, s), 8.09 (1H, d, J=8.0), 8.85 (1H, s); δ_{C} (CDCl₃, 101 MHz): 22.7, 41.6, 51.6, 54.9, 55.3, 126.4, 126.5, 126.6, 126.9, 127.6, 127.7, 128.3, 128.5, 129.0, 133.7, 135.3, 145.2, 147.5, 150.4, 171.6; m/z calculated for C₂₁H₂₂N₂O₂: 334.1683 (M⁺), found 334.1682 (M⁺).

### N-(α-Methylbenzyl)-3-Amino-3-(Isoquin-4-yl)propionic Acid Methyl Ester and N-(α-Methylbenzyl)-3-(Isoquin-4-yl)-Acrylic Amide

To a stirred solution of α-methylbenzylamine (1.21 g; 10.0 mmol) and triethylamine (1.44 g; 14.3 mmol) in dry THF (20 mL) at room temperature under argon was added drop-wise trimethylsilyl chloride (1.28 g; 11.8 mmol). The mixture was allowed to stir at room temperature for 1 h after which triethylamine hydrochloride was removed via filtration under a blanket of argon. The resulting clear silylamine, in dry THF, was cooled to −78° C. and n-butyl lithium (1.6 M in hexanes; 4.69 mL; 7.50 mmol) was added drop-wise and the mixture stirred for 15 min. To this solution was added drop-wise 3-(isoquin4-yl)acrylic acid methyl ester (1.07 g; 5.00 mmol) in dry THF (6 mL). The resulting mixture was stirred at −78° C. for 15 min before quenching with saturated ammonium chloride solution (6 mL). The reaction mixture was allowed to warm to room temperature and extracted with Et₂O (3×25 mL). The combined organic layers were concentrated under reduced pressure, after which 1 N hydrochloric acid (10 mL) was added. The resulting mixture was washed with Et₂O (3×25 mL) and the organic layers discarded. The aqueous layer was made basic with solid potassium carbonate and extracted with DCM (4×25 mL). The combined organic layers were dried over sodium sulfate and concentrated under reduced pressure to give a red oil. Purification by column chromatography using gradient elution with EtOAc:Hexanes (2:1, 3:1, 8:1, 10:1) as the eluent gave a yellow solid, which was recrystallized to give an off-white crystalline solid (Amide: 157 mg; 10%): mp 202-204° C.; R_{f} 0.17 (D); R_{f} 0.49 (I); νₘₐₓ (KBr): 3268, 3056, 1653, 1619, 1546, 974, 751 cm⁻¹; m/z (EI): 302.3, 197.0, 182.0, 128.1, 153.8, 128.1, 120.0, 104.7, 76.9; δ_{H} (CDCl₃, 200 MHz): 1.62 (3H, d, J=7.0), 5.33 (1H, quin, J=7.4), 6.62 (1H, d, J=15.2), 6.82 (1H, d, J=7.8), 7.27-7.45 (5H, m), 7.63 (1H, ddd, J=9.0, 6.8 and 1.2), 7.75 (1H, ddd, J=9.8, 7.0 and 1.6), 7.95 (1H, dd, J=7.8 and 1.0), 8.13 (1H, d, J=8.6), 8.32 (1H, d, J=15.6), 8.71 (1H, s), 9.04 (1H, s); δ_{C} (CDCl₃, 126 MHz): 21.7, 49.1, 122.8, 125.3, 126.4, 126.7, 127.4, 127.6, 128.1, 128.1, 128.7, 131.2, 133.8, 135.0, 140.6, 143.1, 153.2, 164.3; m/z calculated for C₂₀H₁₈N₂O: 302.1419 (M⁺), found 302.1413 (M⁺). Concentration of the filtrate under reduced pressure gave an amber oil (Crude Ester: 77.1 mg; 5%): R_{f} 0.05 (C); R_{f} 0.49 (I); νₘₐₓ (nujol): 3323, 1733, 1674, 1272, 904, 757 cm⁻¹; m/z (EI): 334.2, 319.1, 228.9, 212.9, 182.5, 153.8, 119.7, 104.8, 76.8; δ_{H} (CDCl₃, 200 MHz): 1.38 (3H, d, J=6.2), 2.90 (2H, dd, J=7.6 and 6.4), 3.63 (3H, s), 3.78 (1H, q, J=6.4), 4.94 (1H, dd, J=8.0 and 6.0), 7.21 (5H, s), 7.58-7.72 (2H, m), 7.96 (1H, dd, J=7.4 and 0.6), 8.12 (1H, d, J=8.6), 8.54 (1H, s), 9.12 (1H, s); δ_{C} (CDCl₃, 126 MHz): 22.7, 41.5, 51.6, 52.5, 55.6, 122.5, 126.6, 126.8, 126.9, 127.1, 127.8, 128.0, 128.3, 128.4, 130.4, 131.4, 133.9, 141.6, 145.1, 152.3, 171.9.

### N-(-α-Methylbenzyl)-3-Amino-3-(Benzo[d]furan-2-yl)propionic Acid Methyl Ester

To a stirred solution of α-methylbenzylamine (2.91 g; 24.0 mmol) and triethylamine (3.47 g; 34.3 mmol) in dry THF (30 mL) at room temperature under argon was added drop-wise trimethylsilyl chloride (3.06 g; 28.3 mmol). The mixture was allowed to stir at room temperature for 1 h after which triethylamine hydrochloride was removed via filtration under a blanket of argon. The resulting clear silylamine, in dry THF, was cooled to −78° C. and n-butyl lithium (1.6 M in Hexanes; 11.3 mL; 18.0 mmol) was added drop-wise and the mixture stirred for 15 min. To this solution was added drop-wise 3-(benzo[d]furan-2-yl)acrylic acid methyl ester (2.43 g; 12.0 mmol) in dry THF (7 mL). The resulting mixture was stirred at −78° C. for 15 min before quenching with saturated ammonium chloride solution (12 mL). The reaction mixture was allowed to warm to room temperature and extracted with Et₂O (3×25 mL). The combined organic layers were concentrated under reduced pressure, after which 1 N hydrochloric acid (12 mL) was added. The resulting pale yellow precipitate was removed via filtration, dissolved in DCM (75 mL) and washed with saturated sodium bicarbonate solution (4×25 mL), saturated sodium chloride solution (4×25 mL) and water (4×25 mL). The organic layer was dried over sodium sulfate and concentrated under reduced pressure to give an amber oil. Purification by column chromatography using EtOAc:hexanes (1:2) as the eluent gave a yellow oil (1.23 g; 32%): R_{f} 0.71 (D); R_{f} 0.51 (E); νₘₐₓ (nujol): 3331, 1740, 1255, 1132, 809, 756 cm⁻¹; m/z (EI): 323.2, 308.2, 250.1, 218.0, 204.1, 160.9, 104.8, 76.9; δ_{H} (CDCl₃, 200 MHz): 1.40 (3H, d, J=6.4), 2.89 (2H, d, J=6.8), 3.68 (3H, s), 3.83 (1H, q, J=6.2), 6.57 (1H, s), 7.18-7.31 (7H, m), 7.42-7.46 (1H, m), 7.50-7.55 (1H, m); δ_{C} (CDCl₃, 101 MHz): 23.4, 39.6, 51.3, 52.0, 55.5, 103.8, 111.5, 121.2, 123.0, 123.1, 124.3, 126.9, 127.2, 127.4, 128.6, 128.7, 145.9, 155.1, 158.4, 172.0; m/z calculated for C₂₀H₂₁NO₃: 323.1521 (M⁺), found 323.1512 (M⁺).

### N-(-α-Methylbenzyl)-N′(benzenesulfonyl)-3-(2-methylindol-5-yl)propionic Acid Methyl Ester and N-(-α-Methylbenzyl)-N′(benzenesulfonyl)-3-(2-methylindol-5-yl)acrylic Amide

To a stirred solution of α-methylbenzylamine (0.911 g; 7.50 mmol) and triethylamine (1.07 g; 10.6 mmol) in dry THF (15 mL) at room temperature under argon was added drop-wise trimethylsilyl chloride (0.896 g; 8.85 mmol). The mixture was allowed to stir at room temperature for 1 h after which triethylamine hydrochloride was removed via filtration under a blanket of argon. The resulting clear silylamine, in dry THF, was cooled to −78° C. and n-butyl lithium (1.6 M in Hexanes; 3.53 mL; 5.62 mmol) was added drop-wise and the mixture stirred for 15 min. To this solution was added drop-wise N-(benzenesulfonyl)-3-(2-methylindol-5-yl)acrylic acid methyl ester (1.33 g; 3.75 mmol) in dry THF (6 mL). The resulting mixture was stirred at −78° C. for 1 h before quenching with saturated ammonium chloride solution (10 mL). The reaction mixture was allowed to warm to room temperature and extracted with Et₂O (3×25 mL). The combined organic layers were concentrated under reduced pressure, after which 1 N hydrochloric acid (10 mL) was added. The resulting pale orange precipitate was removed via filtration, dissolved in DCM (50 mL) and washed with saturated sodium bicarbonate solution (4×15 mL), saturated sodium chloride solution (4×15 mL) and water (4×15 mL). The organic layer was dried over sodium sulfate and concentrated under reduced pressure to give an amber oil. Purification by column chromatography using EtOAc:hexanes (1:2) as the eluent gave two products: a yellow oil (Ester: 0.529 g; 30%): R_{f} 0.15 (C); R_{f} 0.68 (I); νₘₐₓ (nujol): 3330, 1735, 1459, 1374, 1165, 1094, 888, 817, 727 cm^{-1; δ}_{H} (CDCl₃, 400 MHz): 1.36 (3H, d, J=6.5), 2.60 (3H, d, J=0.9), 3.60 (3H, s), 3.68 (1H, q, J=8.0), 4.27 (1H, t, J=8.0), 7.18-7.29 (m, 6H), 7.33 (1H, s), 7.45 (2H, t, J=7.36), 7.56 (1H, tt, J=4.8 and 1.6), 7.80 (2H, dd, J=8.2 and 1.0), 8.08 (1H, d, J=8.6); δ_{C} (CDCl₃, 101 MHz): 16.0, 22.6, 42.8, 51.8, 55.0, 57.1, 109.9, 114.8, 123.1, 126.7, 126.7, 126.9, 126.9, 127.2, 128.7, 128.7, 129.6, 129.6, 130.1, 130.1, 134.0, 134.0, 136.6, 138.0, 139.6; m/z calculated for C₂₇H₂₈N₂O₄S: 476.1770 (M⁺), found 476.1765 (M⁺) and a yellow crystalline solid (Amide: 0.248 g; 15%): mp: 84-86° C.; R_{f} 0.08 (C); R_{f} 0.68 (I); νₘₐₓ (KBr): 3272, 3060, 1733, 1658, 1536, 1367, 1170, 982, 636 cm^{-1; δ}_{H} (CDCl₃, 400 MHz): 1.56 (3H, d, J=6.9), 2.57 (3H, d, J=0.9), 5.28 (1H, quin, J=7.2), 6.15 (1H, d, J=7.9), 6.30 (1H, s), 6.43 (1H, d, J=15.6), 7.26-7.44 (8H, m), 7.47 (1H, s), 7.54 (1H, tt, J=7.5 and 1.0), 7.76 (2H, dd, J=8.3 and 1.0), 8.12 (1H, d, J=8.7); δ_{C} (CDCl₃, 101 MHz): 16.0, 22.1, 49.2, 110.0, 115.0, 120.2, 120.5, 123.4, 126.6, 126.6, 127.7, 129.0, 129.0, 129.7, 129.7, 130.3, 130.8, 134.2, 138.0, 139.4, 141.6, 143.5, 165.6; m/z calculated for C₂₆H₂₄N₂O₃S: 444.1508 (M⁺), found 444.1513 (M⁺).

### N-(Benzenesulfonyl)-3-(Indol-5-yl)acrylic Acid Methyl Ester

To a solution of 3-(indol-5-yl)-acrylic acid methyl ester (1.03 g; 5.12 mmol) in dry THF (18 mL) at −78° C. under argon was added drop-wise lithium diisopropylamide, prepared from diisopropylamine (0.529 g; 5.23 mmol) and n-butyl lithium (1.6 M in hexanes; 3.24 mL; 5.12 mmol) in dry THF (2 mL) at −78° C. under argon. The resulting mixture was stirred for 25 min at −78° C. and then quenched with benzenesulfonyl chloride (0.950 g; 5.38 mmol). Upon cooling to room temperature overnight the reaction mixture was cooled to 5° C., poured into 2% (w/v) sodium bicarbonate solution (50 mL) and extracted with Et₂O (3×30 mL). The combined organic layers were washed with 3% (w/v) sodium thiosulfate solution (3×25 mL), distilled water (3×20 mL) and saturated sodium chloride solution (3×25 mL), dried over sodium sulfate and concentrated under reduced pressure to give a yellow solid. Purification by column chromatography with Et₂O:hexanes (2:1) as the eluent afforded a yellow powder (1.26 g; 72%): mp 134-136° C.; R_{f} 0.37 (J); R_{f} 0.79 (E); νₘₐₓ (KBr): 3123, 1717, 1637, 1365, 1308, 1176, 1115; m/z (EI): 341.2, 310.2, 200.1, 185.0, 169.0, 140.9, 115.0, 77.1; δ_{H} (CDCl₃, 200 MHz): 3.79 (3H, s), 6.41 (1H, d, J=16.0), 6.67 (1H, dd, J=3.6 and 0.6), 7.40-7.54 (4H, m), 7.59 (1H, d, J=3.8), 7.66 (1H, d, J=1.4), 7.74 (1H, d, J=16.0), 7.88 (2H, dd, J=8.2 and 1.8), 7.99 (1H, d, J=8.4); δ_{C} (CDCl₃, 126 MHz): 51.5, 109.2, 113.8, 117.0, 121.8, 124.1, 126.7, 127.3, 129.3, 129.8, 131.1, 134.0, 135.7, 138.0, 144.8, 167.4; m/z calculated for C₁₈H₁₅O₄NS: 341.0722 (M⁺), found 341.0718 (M⁺).

### N-(Benzenesulfonyl)-3-(2-Methylindol-5-yl)acrylic Acid Methyl Ester

To a solution of 3-(2-methylindol-5-yl)-acrylic acid methyl ester (1.10 g; 5.12 mmol) in dry THF (20 mL) at −78° C. under argon was added drop-wise lithium diisopropylamide, prepared from diisopropylamine (0.529 g; 5.23 mmol) and n-butyl lithium (1.6 M in hexanes; 3.24 mL; 5.12 mmol) in dry THF (2 mL) at −78° C. under argon. The resulting mixture was stirred for 25 min at −78° C. and then quenched with benzenesulfonyl chloride (0.950 g; 5.38 mmol). Upon cooling to room temperature overnight the reaction mixture was cooled to 5° C., poured into 2% (w/v) sodium bicarbonate solution (50 mL) and extracted with Et₂O (3×30 mL). The combined organic layers were washed with 3% (w/v) sodium thiosulfate solution (3×25 mL), distilled water (3×20 mL) and saturated sodium chloride solution (3×25 mL), dried over sodium sulfate and concentrated under reduced pressure to give a tan solid. Purification by column chromatography with DCM as the eluent afforded a white powder (1.57 g; 86%): mp 126-128° C.; R_{f} 0.39 (C); R_{f} 0.72 (E); νₘₐₓ (KBr): 1725, 1635, 1367, 1281, 1242, 1169, 994, 640; m/z (EI): 355.3, 214.2, 199.0, 182.0, 153.8, 140.8; δ_{H} (CDCl₃, 400 MHz): 2.60 (3H, s), 3.81 (3H, s), 6.37 (1H, s), 6.43 (1H, d, J=16.0), 7.43 (1H, d, J=1.7), 7.45 (2H, d, J=8.0), 7.56 (2H, tt, J=7.5, 1.2), 7.75 (1H, d, J=16.3), 7.79 (2H, dd, J=8.4, 1.1), 8.17 (1H, d, J=8.7); δ_{C} (CDCl₃, 101 MHz): 16.0, 52.0, 109.0, 115.1, 117.1, 120.7, 123.8, 126.6, 129.7, 130.2, 130.4, 134.2, 138.4, 138.9, 139.4, 145.4, 167.9; m/z calculated for C₁₉H₁₇NO₄S: 355.0878 (M⁺), found 355.0875 (M⁺).

### N-(o-Methoxyphenyl)quinoline-2-Carboximine

A mixture of quinoline-2-carboxaldehyde (3.14 g; 20.0 mmol) and o-anisidine (2.46 g; 20.0 mmol) in DCM (30 mL) was allowed to stir over 3 Å molecular sieves at room temperature overnight. The reaction mixture was filtered through Celite® and concentrated under reduced pressure to give an amber solid. Purification by recrystallization with EtOAc and hexanes gave a mustard powder (2.35 g; 45%): mp 109-111° C.; R_{f} 0.35 (C); R_{f} 0.45 (E); νₘₐₓ (KBr): 3422, 1587, 1242, 1023, 746; m/z (EI): 262.2, 231.1, 154.7, 27.9, 91.7, 76.9; δ_{H} (CDCl₃, 400 MHz): 3.93 (3H, s), 7.00-7.04 (2H, m), 7.19 (1H, dd, J=7.6 and 1.4), 7.26 (1H, td, J=7.9 and 1.6), 7.60 (1H, t, J=7.1), 7.76 (1H, t, J=8.3), 7.86 (1H, d, J=8.1), 8.17 (1H, d, J=8.5), 8.25 (1H, d, J=8.6), 8.43 (1H, d, J=8.6), 8.85 (1H, s); δ_{C} (CDCl₃, 101 MHz):56.2, 112.0, 119.2, 120.9, 121.4, 128.0, 128.1, 128.1, 129.2, 130.0, 130.2, 136.9, 140.7, 148.3, 152.9, 155.3, 162.1; m/z calculated for C₁₇H₁₄N₂O: 262.1106 (M⁺), found 262.1100 (M⁺).

### N-(o-Methoxyphenyl)-2-Chloroquinoline-3-Carboximine

A mixture of 2-chloroquinoline-3-carboxaldehyde (3.85 g; 20.0 mmol) and o-anisidine (2.46 g; 20.0 mmol) in DCM (30 mL) was allowed to stir over 3 Å molecular sieves at room temperature overnight. The reaction mixture was filtered through Celite® and concentrated under reduced pressure to give an amber solid. Purification by recrystallization with EtOAc and hexanes gave a bright yellow powder (4.80 g; 81%): mp 133-135° C.; R_{f} 0.41 (C); R_{f} 0.60 (E); νₘₐₓ (KBr): 3414, 1580, 1245, 1047, 1020; m/z (EI): 296.1, 261.1, 245.1, 231.0, 162.8, 133.9, 119.9, 91.7, 76.7; δ_{H} (CDCl₃, 400 MHz): 3.94 (3H, s), 7.00-7.05 (2H, s), 7.12 (1H, dd, J=7.6 and 1.7), 7.28 (1H, td, J=8.2 and 1.7), 7.60 (1H, ddd, J=8.1, 7.0 and 1.1), 7.80 (1H, ddd, J=8.4, 7.0 and 1.4), 7.96 (1H, dt, J=8.2 and 0.8), 8.05 (1H, dd, J=8.5 and 0.6), 9.09 (1H, s), 9.03 (1H, s); δ_{C} (CDCl₃, 101 MHz): 52.6, 111.9, 120.9, 121.5, 127.5, 127.9, 128.0, 128.1, 128.7, 129.2, 132.2, 138.2, 141.3, 148.8, 150.6, 152.8, 157.1; m/z calculated for C₁₇H₁₃N₂OCl: 296.0716 (M⁺), found 269.0727 (M⁺).

### N-(o-Methoxyphenyl)-3-Amino-3-(Quinolin-2-yl)propionic Acid Methyl Ester

To a solution of N-(o-methoxyphenyl)quinoline-2-carboximine (0.53 g; 2.01 mmol) and methyl bromoacetate (0.74 g; 4.80 mmol) in DCM (8 mL) under argon at room temperature was added zinc-copper couple (0.54 g; 8.00 mmol). The reaction mixture was allowed to stir for 17 hr at room temperature after which it was poured into 1 N hydrochloric acid solution (30 mL). The aqueous layer was extracted with DCM (1×25 mL). The combined organic layers were washed with saturated sodium bicarbonate solution (2×25 mL), water (2×25 mL) and saturated sodium chloride solution (2×25 mL). The organic layer was dried over sodium sulfate and concentrated under reduced pressure to give an amber oil. Purification by column chromatography using EtOAc:hexanes (1:2) as the eluent gave a pale yellow crystalline solid (0.32 g; 47%): mp 119-121° C.; R_{f} 0.33 (C); R_{f} 0.32 (E); νₘₐₓ (KBr): 3385, 1727, 1598, 1281, 1230, 1182, 1048, 1023, 913; m/z (EI): 336.2, 305.2, 263.1, 247.1, 232.1, 182.0, 155.9, 142.9, 128.9, 107.9; δ_{H} (CDCl₃, 400 MHz): 3.17 (2H, ddd, J=12.7, 7.2 and 5.9), 3.67 (3H, s), 3.91 (3H, s), 5.26 (1H, t, J=5.9), 6.60 (1H, dd, J=7.8 and 1.5), 6.67 (1H, td, J=7.7 and 1.5), 6.76 (1H, td, J=7.6 and 1.4), 6.80 (1H, dd, J=7.8 and 1.2), 7.53 (1H, d, J=7.1), 7.58 (1H, d, J=8.8), 7.74 (1H, t, J=8.2), 7.80 (1H, d, J=8.1), 8.12 (1H, d, J=8.5), 8.18 (1H, d, J=7.9); δ_{C} (CDCl₃, 101 MHz): 40.6, 52.1, 55.9, 56.1, 110.1, 111.4, 117.6, 119.8, 121.5, 126.9, 127.8, 127.9, 129.0, 130.2, 136.8, 137.9, 147.5, 162.2, 172.2; m/z calculated for C₂₀H₂₀N₂O₃: 336.1474 (M⁺), found 336.1468 (M⁺). Preparation of Zinc-Copper Couple: To a vigorously stirred solution of cupric acetate monohydrate (0.3 g; 1.5 mmol) in glacial acetic acid (5 mL) at high temperature was added portion-wise zinc dust (3 g; 46 mmol). The reaction mixture was allowed to stir for 30 min after which the glacial acetic acid was decanted. The couple was washed with glacial acetic acid (1×10 mL), Et₂O (1×10 mL) and benzene (1×10 mL). The residue solvent was removed under a stream of argon to give a dark gray powder.

### N-(o-Methoxyphenyl)-3-Amino-3-(2-Chloroquinolin-3-yl)propionic Acid Methyl Ester

To a solution of N-(o-methoxyphenyl)-2-chloroquinoline-3-carboximine (0.60 g; 2.01 mmol) and methyl bromoacetate (0.74 g; 4.80 mmol) in DCM (8 mL) under argon at room temperature was added zinc-copper couple (0.54 g; 8.00 mmol). The reaction mixture was allowed to stir for 17 hr at room temperature after which it was poured into 1 N hydrochloric acid solution (30 mL). The aqueous layer was extracted with DCM (1×25 mL). The combined organic layers were washed with saturated sodium bicarbonate solution (2×25 mL), water (2×25 mL) and saturated sodium chloride solution (2×25 mL). The organic layer was dried over sodium sulfate and concentrated under reduced pressure to give a brown solid. Purification by filtration through a silica plug using EtOAc:hexanes (1:2) as the eluent gave a pale yellow crystalline solid (0.32 g; 43%): mp 135-137° C.; R_{f} 0.32 (C); R_{f} 0.55 (E); νₘₐₓ (KBr): 3374, 1732, 1596, 1290, 1255, 1203, 1060, 1008, 954; m/z (EI): 370.2, 297.1, 231.0, 189.9, 175.9, 162.9, 126.9, 107.9; δ_{H} (CDCl₃, 400 MHz): 3.15 (2H, dd, J=13.6 and 4.2), 3.68 (3H, s), 3.96 (3H, s), 5.33 (1H, q, J=4.2), 6.32-6.35 (1H, m), 668-6.71 (2H, m), 6.80-6.83 (1H, m), 7.51 (1H, ddd, J=8.1, 7.0 and 1.1), 7.70 (1H, ddd, J=8.4, 7.0 and 1.4), 7.76 (1H, dd, J=8.2 and 0.9), 8.01 (1H, d, J=8.5), 8.29 (1H, s); δ_{C} (CDCl₃, 101 MHz): 40.4, 52.3, 52.4, 56.0, 110.1, 112.2, 118.6, 121.5, 127.5, 127.7, 128.2, 128.4, 130.8, 133.0, 137.1, 147.4, 147.7, 149.8, 171.4; m/z calculated for C₂₀H₁₉N₂O₃Cl: 370.1084 (M⁺), found 370.1087 (M⁺).

### 3-Amino-3-(2-Chloroquinolin-3-yl)propionic Acid Methyl Ester

To a stirred solution of N-(o-methoxyphenyl)-3-amino-3-(2-chloroquinolin-3-yl)propionic acid methyl ester (0.823 g; 2.22 mmol) in MeCN—H₂O (5:1; 22 mL) at room temperature was added ceric ammonium nitrate (4.87 g; 8.88 mmol). The reaction mixture was allowed to stir at room temperature for 17 h after which it was quenched with saturated sodium bicarbonate solution (4 mL). The mixture was stirred for 15 min and the resulting solid removed via filtration. The filtrated was extracted with Et₂O (4×30 mL). The combined organic layers were dried over sodium sulfate and concentrated under reduced pressure to give a red oil. Purification by column chromatography using Et₂O:hexanes as the eluent gave a pale yellow powder (109 mg; 19%): mp 103-106° C.; R_{f} 0.53 (D); R_{f} 0.28 (J); νₘₐₓ (KBr): 3469, 3250, 1746, 1664, 1245, 1083, 753 cm⁻¹; m/z (EI): 265.9, 229.6, 191.9, 161.9, 127.9; δ_{H} (CDCl₃, 400 MHz): 2.60 (1H, dd, J=16.6 and 9.6), 3.02 (1H, dd, J=16.6 and 2.6), 3.73 (3H, s), 5.54 (1H, dd, J=9.6 and 2.0), 7.53 (1H, ddd, J=8.1, 7.0 and 1.1), 7.69 (1H, ddd, J=8.4, 7.0 and 1.4), 7.80 (1H, d, J=8.2), 7.97 (1H, d, J=8.6), 8.42 (1H, s); δ_{C} (CDCl₃, 101 MHz): 41.6, 52.4, 67.1, 127.6, 127.9, 128.1, 128.3, 130.8, 134.6, 136.5, 147.2, 148.3, 172.9; m/z calculated for C₁₃H₁₃N₂O₂Cl: 265.0506 (M⁺), found 265.0511 (M⁺).

### N-(o-Methoxyphenyl)-3-Amino-3-(Benzo[d]thiophen-3-yl)propionic Acid Methyl Ester

A mixture of benzo[d]thiophene-3-carboxaldehyde (1.09 g; 6.70 mmol) and o-anisidine (0.827 g; 6.70 mmol) in DCM (30 mL) was allowed to stir over 3 Å molecular sieves at room temperature overnight. The reaction mixture was filtered through Celite® and concentrated under reduced pressure to give N-(o-methoxyphenyl)benzo[d]thiophene-3-carboximine an amber oil. To this oil was added DCM (30 mL), methyl bromoacetate (2.2 g; 14.4 mmol) and zinc-copper couple (1.32 g; 19.6 mmol). The resulting mixture was allowed to stir at room temperature under argon for 17 h after which it was poured into 1 N hydrochloric acid solution (60 mL). The aqueous layer was extracted with DCM (1×50 mL). The combined organic layers were washed with saturated sodium bicarbonate solution (2×50 mL), water (2×50 mL) and saturated sodium chloride solution (2×50 mL). The organic layer was dried over sodium sulfate and concentrated under reduced pressure to give a brown oil. Purification by column chromatography using EtOAc:hexanes (1:4) as the eluent gave a yellow oil (1.26 g; 55%): R_{f} 0.43 (C); R_{f} 0.68 (E); νₘₐₓ (nujol): 3415, 1743, 1602, 1248, 1216, 1027, 761 cm⁻¹; m/z (EI): 341.1, 304.3, 219.2, 177.2, 160.2; δ_{H} (CDCl₃, 400 MHz): 3.00 (1H, dd, J=15.2 and 7.6), 3.10 (1H, dd, J=15.2 and 9.5), 3.69 (3H, s), 3.89 (3H, s), 5.33 (1H, t, J=6.3), 6.58 (1H, dd, J=7.7 and 1.4), 6.70 (1H, td, J=7.7 and 1.6), 6.79 (2H, ddd, J=15.9, 7.6 and 1.5), 7.37-7.45 (3H, m), 7.87 (2H, dd, J=7.9 and 1.1); δ_{C} (CDCl₃, 101 MHz): 40.8, 50.2, 52.3, 55.9, 110.0, 111.5, 117.7, 121.6, 121.9, 122.7, 123.4, 124.6, 124.8, 136.8, 137.6, 141.5, 147.4, 171.9; m/z calculated for C₁₉H₁₉NO₃S: 341.1086 (M⁺), found 341.1101 (M⁺).

### N-(o-Methoxyphenyl)-3-Amino-3-(Benzo[d]furan-2-yl)propionic Acid Methyl Ester

To a solution of N-(o-methoxyphenyl)benzo[d]furan-2-carboximine (1.51 g; 6.01 mmol) and methyl bromoacetate (2.22 g; 14.4 mmol) in DCM (20 mL) under argon at room temperature was added zinc-copper couple (1.21 g; 18.0 mmol). The reaction mixture was allowed to stir for 16 hr at room temperature after which it was poured into 1 N hydrochloric acid solution (60 mL). The aqueous layer was extracted with DCM (1×50 mL). The combined organic layers were washed with saturated sodium bicarbonate solution (2×50 mL), water (2×50 mL) and saturated sodium chloride solution (2×50 mL). The organic layer was dried over sodium sulfate and concentrated under reduced pressure to give a brown oil. Purification by column chromatography using EtOAc:hexanes (1:4) as the eluent gave a pale yellow crystalline solid (0.382 g; 20%): mp ° C.; R_{f} 0.31 (C); R_{f} 0.68 (E); νₘₐₓ (KBr): 3363, 1724, 1597, 1255, 1225, 1023 cm⁻¹; m/z (EI): 325.2, 252.1, 203.2, 161.1, 144.1; δ_{H} (CDCl₃, 400 MHz): 3.04 (1H, dd, J=15.5 and 7.0), 3.10 (1H, dd, J=15.5 and 6.1), 3.70 (3H, s), 3.89 (3H, s), 5.18 (1H, t, J=6.5), 6.62 (1H, s), 6.71-6.76 (2H, m), 6.81-6.87 (2H, m), 7.20 (1H, td, J=7.4 and 1.0), 7.24-7.28 (1H, m), 7.45 (1H, dd, J=8.2 and 0.5), 7.49 (1H, dd, J=7.7 and 0.8); δ_{C} (CDCl₃, 101 MHz): 39.5, 49.5, 52.3, 55.9, 103.7, 110.2, 111.5, 111.7, 118.2, 121.3, 121.5, 123.0, 124.3, 128.6, 136.4, 147.6, 155.2, 157.6, 171.5; m/z calculated for C₁₉H₁₉NO₄: 325.1314 (M⁺), found 325.1321 (M⁺).

### Example 2

### Biological Analysis of Some Compounds of the Invention

The MES and PTZ assays were performed by the Anticonvulsant Drug Development (ADD) Program in the Epilepsy Branch of the NIH (see, e.g., Stables and Kupferberg (1997) The NIH anticonvulsant Drug Development (ADD) Program: Preclinical Anticonvulsant Screening Project, Libby & Sons). All compounds were tested with either male Carworth Farms #1 mice or male Sprague-Dawley rats. Each test compound was administered via an i.p. injection at 300, 100, and 30 mg/kg.

Pilocarpine Assay

A seizure model is performed using adult male Sprague-Dawley rats in accordance with the guidelines of the Canada Council on Animal Care and under the supervision of the Queen's University Animal Ethics Committee. This test procedure was adopted from previous work by Turski et al. (1984) Brain Res. 321:237. The test compounds are administered at 100 mg/kg by intraperitoneal (i.p.) injection. Seizures are induced 20 minutes afterwards by i.p. administration of pilocarpine hydrochloride (350 mg/kg). Protection is defined as the absence of clonic spasms over a 30 minute observation period after pilocarpine administration.

MES Induced Seizure Model

For the maximal electroshock seizure test (MES), corneal electrodes primed with a drop of electrolyte solution (0.9% NaCl) are applied to the eyes of the animal and an electrical stimulus (50 mA for mice, 150 mA for rats; 60 Hz) is delivered for 0.2 seconds at 0.5 and 4.0 hours after test compound administration. The animals are restrained by hand and are released at the moment of stimulation in order to permit observation of the seizure. Abolition of hind-leg tonic-extensor component (hind-leg tonic extension does not exceed a 90° angle to the plane of the body) indicates that the compound prevents MES-induced seizure spread.

PTZ Induced Seizure Model

In the subcutaneous pentylenetetrazole (PTZ)-induced seizure model, seizures are induced 0.5 and 4 hrs after test compound administration by i.p. injection of PTZ (85 mg/kg in mice and 70 mg/kg in rats). Protection is defined as the inhibition of clonic spasms over a 30 min observation period.

SRS Model of Epilepsy

The “spontaneous recurrent seizures” (SRS) model of epilepsy is used to evaluate candidate compounds in a model for Phase 1 epileptogenesis (see, e.g., Mello, E. et al., Epilepsia (1993) 34:985; Cavalheiro, J. et al., Epilepsia (1991) 32:778). In the SRS model, an adult male Sprague-Dawley rat (c. 260 g) is given pilocarpine by injection (380 mg/kg i.p.). Within 25 minutes, the animal enters status epilepticus, which lasts for 3 hours, and then diazepam is administered to stop seizures. The rat is allowed to spontaneously recover and is given food and water ad lib. and maintained on a 12 hour/12 hour light/dusk cycle. Beginning on about day 13-15, the rats develop spontaneous recurrent seizures, which occur at the rate of about 4-5 per week. The rats are videotaped 8 hours per day 5 days per week, and the videotapes are reviewed for behavioral seizures (including head nodding, forelimb clonus, and rearing), which are counted. The animals are watched for two months, permitting evaluation of a sufficient number of seizures. An experimental compound for evaluation can be administered at either of two times: Time 1, on Day 1, after the cessation of status epilepticus but before the onset of SRS; or Time 2, on Day 30, when the rats have been experiencing SRS for about two weeks. Administration of the candidate compound at Time 1 permits evaluation for anti-epileptogenic properties (ability to prevent the onset of seizures); administration of compounds at Time 2 permits evaluation of drugs as anti-ictogenics with the ability to suppress established seizures.

As a reference, the standard anticonvulsant phenyloin was administered (20 mg/kg/day i.v. for 10 day) at either Time 1 or Time 2. As expected, phenyloin was ineffective in preventing the onset of seizures when administered at Time 1, but was 75% effective at decreasing seizure frequency by 50% or more when administered at Time 2.

TABLE 2
Efficacy of Some Compounds of The Invention in The Assays Described Above

| | Anti-Ictogenic Assays | | | | |
|---|---|---|---|---|---|
| | MES | | PTZ | | |
| Compound | 0.5 hr | 4 hr | 0.5 hr | 4 hr | Pilocarpine |
| 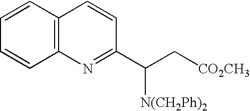 | — | 1/3 @ 100 mg | — | — | 4s (Inactive) |
| 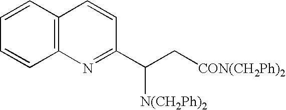 | — | — | — | — | 3s (Min. Active) |
| 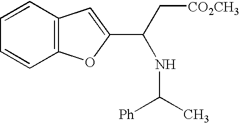 | — | — | 2/5 @ 300 mg | 1/5 @100 mg | 2s (Active) |
| 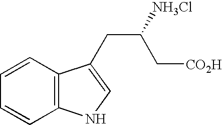 | — | — | — | — | 1s (Active) |
| 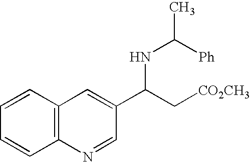 | — | — | — | — | 3s (Min. Active) |
| 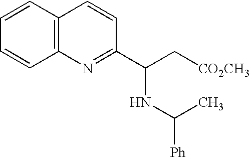 | — | — | — | — | 2s (Active) |

| | | | |
|---|---|---|---|
| 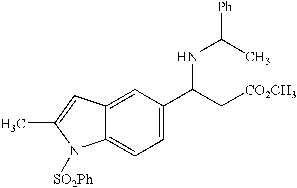 | RP | RP | 1s (Active) |
| 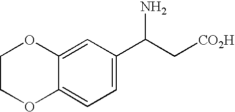 | RP | RP | 3s (Min. Active) |
| 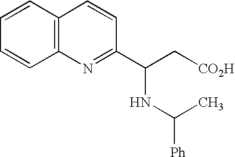 | RP | RP | 2s (Active) |
| 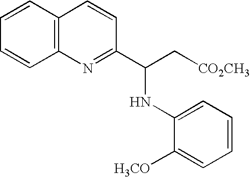 | RP | RP | 2s (Active) |
| 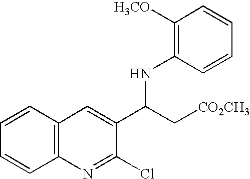 | RP | RP | 3s (Min. Active) |
| 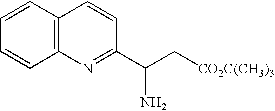 | RP | RP | RP |
| 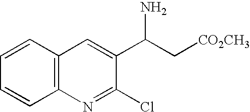 | RP | RP | RP |
| 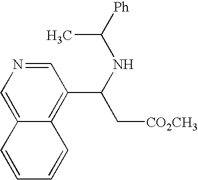 | RP | RP | RP |
| 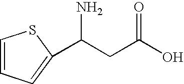 | — | — | 2s (Active) |

— = no activity;
RP = Biological Results Pending

Those skilled in the art will recognize, or be able to ascertain using no more than routine experimentation, numerous equivalents to the specific procedures described herein. Such equivalents are considered to be within the scope of this invention and are covered by the following claims. The contents of all publications cited herein are hereby incorporated by reference.

1. A method for inhibiting epileptogenesis in a subject, comprising administering to said subject an effective amount of an anti-epileptogenic agent, such that said epileptogenesis in said subject is inhibited, wherein said anti-epileptogenic agent is a β-heterocyclic-β-amino acid, or a salt or ester, N-substituted analog, C-substituted analog, bioisostere, or prodrug thereof.

2. A method for treating a subject suffering from an epileptogenesis-associated condition, comprising administering to said subject an effective amount of an anti-epileptogenic agent, such that said subject is treated wherein said anti-epileptogenic agent is a β-heterocyclic-β-amino acid, or a salt or ester, N-substituted analog, C-substituted analog, bioisostere, or prodrug thereof.

3. A method for treating convulsions in a subject comprising administering to said subject an effective amount of an anti-epileptogenic agent, such that said subject is treated, wherein said anti-epileptogenic agent is a β-heterocyclic-β-amino acid, or a salt or ester, N-substituted analog, C-substituted analog, bioisostere, or prodrug thereof.

4. The method of claim 1, wherein said subject is a human who is suffering from head trauma, stroke, schizophrenia, multiple sclerosis, amyotrophic lateral sclerosis, psychosis, cerebral ischemia, Huntington's chorea, motor neuron disease, Alzheimer's disease, dementia, or epilepsy.

5. A method for inhibiting epileptogenesis in a subject, comprising administering to said subject an effective amount of an anti-epileptogenic agent such that said epileptogenesis is inhibited, wherein said anti-epileptogenic agent is of the Formula: 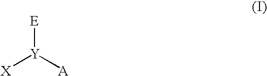
wherein:
X is a heterocyclic moiety;
E is a hydrogen bond donor;
Y is a connecting moiety; and
A is hydrogen bond acceptor,
or a pharmaceutically acceptable salt or ester, N-substituted analog, C-substituted analog, bioisostere, or prodrug thereof.

6. A method for treating an epileptogenesis-associated condition in a subject, comprising administering to said subject an effective amount of an anti-epileptogenic agent such that said subject is treated for said epileptogenesis-associated condition, wherein said anti-epileptogenic agent is of the Formula: 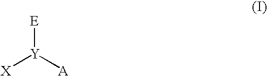
wherein
X is a heterocyclic moiety;
Y is a connecting moiety;
E is a hydrogen bond donor; and
A is hydrogen bond acceptor,
or a pharmaceutically acceptable salt or ester, N-substituted analog, C-substituted analog, bioisostere, or prodrug thereof.

7. A method for treating convulsions in a subject, comprising administering to said subject an effective amount of an anti-epileptogenic agent such that said subject is treated for said convulsions, wherein said anti-epileptogenic agent is of the Formula: 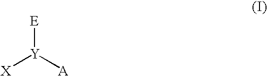
wherein
X is a heterocyclic moiety;
Y is a connecting moiety;
E is a hydrogen bond donor; and
A is hydrogen bond acceptor,
or a pharmaceutically acceptable salt or ester, N-substituted analog, C-substituted analog, bioisostere, or prodrug thereof.

8. The method of claim 5, wherein said connecting moiety is alkyl.

9. The method of claim 7, wherein said anti-epileptogenic agent is of the Formula:

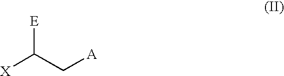

10. The method of claim 9, wherein said hydrogen bond donor is NR²R³, OH, or SH, wherein R² and R³ are each independently hydrogen, alkyl, alkenyl, alkynyl, cycloalkyl, aryl, arylalkyl, alkylcarbonyl, arylcarbonyl, alkoxycarbonyl, or aryloxycarbonyl.

11. The method of claim 10, wherein said hydrogen bond donor is NR²R³.

12. (canceled)

13. The method of claim 9, wherein said hydrogen bond acceptor is carboxylate, carboxylic acid, sulfate, sulfonate, sulfinate, sulfamate, phosphate, phosphonate, tetrazolyl, phosphinate, or phosphorothioate.

14. The method of claim 13, wherein said hydrogen bond acceptor is carboxylate or a carboxylic acid.

15. The method of claim 9, wherein said heterocyclic moiety comprises a heteroaromatic group.

16. The method of claim 15, wherein said heterocyclic moiety comprises a substituted or unsubstituted heterocycle.

17. (canceled)

18. The method of claim 16, wherein said heterocycle is unsubstituted.

19. The method of claim 16, wherein said heterocyclic moiety is multicyclic or polycyclic.

20. The method of claim 19, wherein said heterocyclic moiety comprises two or more bridged or fused rings.

21. The method of claim 20, wherein at least one of said bridged rings is phenyl.

22. The method of claim 20, wherein at least one of said rings is pyridinyl, thienyl, pyrrolyl, pyrimidyl, pyrazinyl, pyrazolyl, oxazolyl, isooxazolyl, thiazolyl, isothiazolyl, imidazolyl, or furanyl.

23. The method of claim 19, wherein said heterocyclic moiety comprises one or more fused rings.

24. The method of claim 23, wherein said heterocyclic moiety comprises one or more aromatic rings.

25. The method of claim 24, wherein said heterocyclic moiety is bicyclic.

26. The method of claim 25, wherein said heterocyclic moiety is benzothiazolonyl, indolonyl, benzooxoazolinyl, benzothiophenyl, benzofuranyl, quinolinyl, isoquinolinyl, benzodioxazolyl, benzoxazolyl, benzothiazolyl, benzoimidazolyl, methylenedioxyphenyl, ethylenedioxyphenyl, indolyl, purinyl, or deazapurinyl.

27. The method of claim 26, wherein said heterocyclic moiety is indolyl, isoquinolyl, quinolinyl, benzothiazolinonyl, benzothiophenyl, benzofuranyl, methylenedioxyphenyl, ethylenedioxyphenyl, or isooxazolylphenyl.

28. (canceled)

29. The method of claim 5, wherein said anti-epileptogenic agent is selected from the group consisting of:
3-(benzo[d]thiophen-3-yl)-3-aminopropionic acid;
3-(benzo[d]furan-2-yl)-3-aminopropionic acid;
N-(1-phenyl-ethyl)-3-(benzo[d]furan-2-yl)-3-aminopropionic acid methyl ester;
3-(benzo[d]dioxolan-5-yl)-3-aminopropionic acid;
3-(quinolin-2-yl)-3-aminopropionic acid;
3-(2-chloroquinolin-3-yl)-3-aminopropionic acid;
3-(benzo[e]dioxan-6-yl)-3-aminopropionic acid;
3-(indol-5-yl)-3-aminopropionic acid;
3-(2-methylindol-5-yl)-aminopropionic acid;
3-(isoquinolin-4-yl)-3-aminopropionic acid;
3-(quinolin-3-yl)-3-aminopropionic acid;
3-(benzo[d]thiazolinon-5-yl)-3-aminopropionic acid; and
3-(4-hydroxy-3-isoxazol-5-ylphenyl)-3-aminopropionic acid
or a pharmaceutically acceptable salt or ester, N-substituted analog, C-substituted analog, bioisostere, or prodrug thereof.

30-41. (canceled)

42. The method of claim 1, wherein said subject is a human who is suffering from pain or anxiety.

43. The method of claim 5, wherein said subject is a human who is suffering from head trauma, stroke, schizophrenia, multiple sclerosis, amyotrophic lateral sclerosis, psychosis, cerebral ischemia, Huntington's chorea, motor neuron disease, Alzheimer's disease, dementia, or epilepsy.
